(19) United States
(12) Patent Application Publication
(10) Pub. No.: US 2019/0142729 A1
(43) Pub. Date: May 16, 2019

(54) COMPOSITIONS AND METHODS FOR TREATING KERATINOUS SUBSTRATES

(71) Applicant: L'OREAL, Paris (FR)

(72) Inventors: Aditi GOGINENI, Rahway, NJ (US); Charles Michael Sanford SHAW, Madison, NJ (US); Nghi VAN NGUYEN, Edison, NJ (US); Jim Mitchell SINGER, South Orange, NJ (US); XianZhi ZHOU, Millburn, NJ (US); Siliu TAN, Westfield, NJ (US); Andrea Michelle ELSEN-WAHRER, Clark, NJ (US)

(21) Appl. No.: 16/243,940

(22) Filed: Jan. 9, 2019

Related U.S. Application Data
(63) Continuation-in-part of application No. 15/396,119, filed on Dec. 30, 2016, now abandoned.
Application No. 15/396,119 is a continuation-in-part of application No. PCT/US2016/069553, filed on Dec. 30, 2016.
Application No. PCT/US2016/069553 is a continuation-in-part of application No. 14/986,370, filed on Dec. 31, 2015.
Application No. 14/986,370 is a continuation-in-part of application No. 14/986,385, filed on Dec. 31, 2015.
Application No. 14/986,385 is a continuation-in-part of application No. 14/986,047, filed on Dec. 31, 2015.

(51) Int. Cl. A61K 8/84; A61K 8/36; A61Q 5/06; A45D 7/06

(52) U.S. Cl. CPC A61K 8/84; A61K 8/361; A61Q 5/06; A61K 2800/884; A61K 2800/5426; A61K 2800/882; A45D 7/06

(57) ABSTRACT

Compositions including polycarbodiimide and derivatives thereof together with at least one additional active selected from one or more a carboxylic acid compound chosen from fatty acids, their salts, and mixtures thereof, a polycarboxylic acid compound other than one that is chosen from fatty acids, their salts, and mixtures thereof, and a cationic polymer to enhance the quality of the keratinous substrates. The present invention relates to a cosmetic treatment and process for treating keratinous materials, in particular for hair-care and hair-styling, wherein the composition can include the polycarbodiimide and at least one additional active in amounts sufficient to impart hydrophobicity to keratinous substrates, including hair fibers, upon application thereto. The present invention, in particular, relates to a hair treatment composition and process for enhancing durable non-permanent shaping and curling of the hair, retention of shaping under humidity, and resistance to frizz.

### CROSS-REFERENCE TO RELATED APPLICATIONS

This patent application claims benefit of U.S. patent application Ser. No. 15/396,119, filed on Dec. 30, 2016, entitled “COMPOSITIONS AND METHODS FOR TREATING KERATINOUS SUBSTRATES,” U.S. patent application Ser. No. 14/986,370, filed on Dec. 31, 2015, entitled “COMPOSITIONS AND METHODS FOR TREATING KERATINOUS SUBSTRATES,” U.S. Pat. No. 14,986,385 entitled “COMPOSITIONS AND METHODS FOR TREATING KERATINOUS SUBSTRATES,” U.S. Pat. No. 14,986,047, entitled “COMPOSITIONS CONTAINING POLYCARBODIIMIDES FOR TREATING KERATINOUS SUBSTRATES,” and PCT Application No. PCT/US2016/069553 filed on Dec. 30, 2016, entitled “COMPOSITIONS CONTAINING POLYCARBODIIMIDES FOR TREATING KERATINOUS SUBSTRATES,” the disclosures of which are incorporated by reference as if fully rewritten herein.

### FIELD OF THE INVENTION

The present invention generally relates to compositions and methods for treating keratinous substrates. More particularly, the present invention relates to keratinous treatment compositions having a polycarbodiimide compound, and, in the various embodiments, at least one additional active selected from one or more a carboxylic acid compound chosen from fatty acids, their salts, and mixtures thereof, a polycarboxylic acid compound other than one that is chosen from fatty acids, their salts, and mixtures thereof, and a cationic polymer, the combination of actives used for one or more of durable non-permanent shaping or for durable retention of a non-permanent shape of at least one keratinous fiber, providing protection from extrinsic damage or repair of keratinous fibers affected by heat, UV radiation/or chemical damage.

### BACKGROUND OF THE INVENTION

The appearance and/or condition of keratinous substrates, for example, keratinous fibers such as hair, skin, nails, and lips, are often affected by both extrinsic and intrinsic factors such as aging. In particular, when keratinous substrates are exposed to environmental conditions, for example, high or low humidity or to ultraviolet radiation from the sun, these substrates can lose many of their desirable properties and even become damaged. Keratinous fibers, especially hair, are constantly exposed to harsh extrinsic conditions, such as sun, chemical damage, e.g., from detergents, bleaching, relaxing, dyeing, and permanent waving, heat, e.g., from hair dryers or curlers, and mechanical stress or wear, e.g., from brushing or grooming activities. In addition, any type of hair can diminish in quality and/or quantity over time by age and/or due to factors such as natural greasiness, sweat, shedded skin cells from the scalp, pollution, dirt, and extreme humidity conditions.

The above-described factors can result in thinning hair and/or harm the visual appearance and the feel of the hair, and lead to lank body and decreased volume. For example, hair can dry out and lose its shine or color or become frizzy and less manageable under low and high humidity conditions. Under low humidity conditions, hair can dry out and dried-out hair tends to be less shiny and more brittle. Conversely, under high humidity conditions, hair tends to absorb water, causing hair to lose its shape and become unmanageable and unattractive. Furthermore, hair can lose its desirable attributes due to physical stress on the hair such as brushing and application of heat. The magnitude of the consequences of these factors is variable, depending on, for example, the quality of the hair, length, style, and environmental factors. As such, these factors generally result in damage to the keratinous fibers, either by affecting protective materials on the surface of the hair (the cuticle), or by altering the hair fiber internally (the cortex).

More specifically, extrinsic conditions may strip protective materials from the surface of the hair, and/or they may disrupt the organized structure of the hair fibers, called the α-structure, which may be accompanied by a decrease in the tensile strength. Such damage to hair by extrinsic factors is more evident the further the hair fiber has grown from the root, because the hair has been exposed longer to such extrinsic factors. In effect, the hair has what may be called a “damage history” as it grows, i.e., the further from the root, the lower the tensile strength and the greater the breakdown in α-structure that has occurred. As a result, consumers continue to seek products such as hair care and hair cosmetic compositions which protect and enhance the appearance of hair as well as reduce the deleterious effects of adverse environmental conditions, photo-damage, and physical stress.

Morphologically, a hair fiber contains four structural units: cuticle, cortex, medulla, and intercellular cement. Robbins, C. R. Chemical and Physical Behavior of Human Hair, 3rd Edition, Springer-Verlag (1994). The cuticle layers are located on the hair surface and consist of flat overlapping cells (“scales”). These scales are attached at the root end and point toward the distal (tip) end of the fiber and form layers around the hair cortex. The cortex comprises the major part of the hair fiber. The cortex consists of spindle-shaped cells, or macrofibrils, that are aligned along the fiber axis. The macrofibrils further consist of microfibrils (highly organized protein units) that are embedded in the matrix of amorphous protein structure. The medulla is a porous region in the center of the fiber. The medulla is a common part of wool fibers but is found only in thicker human hair fibers. Finally, the intercellular cement is the material that binds the cells together, forming the major pathway for diffusion into the fibers.

The mechanical properties of hair are determined by the cortex. A two-phase model for the cortex organization has been suggested. Milczarek et al, Colloid Polym. Sci., 270, 1106-1115 (1992). In this model, water-impenetrable microfilaments (“rods”) are oriented parallel with the fiber axis. The microfilaments are embedded in a water-penetrable matrix (“cement”). Within the microfilaments, coiled protein molecules are arranged in a specific and highly organized way, representing a degree of crystallinity in the hair fiber.

Similar to other crystalline structures, hair fibers display a distinct diffraction pattern when examined by wide-angle X-ray diffraction. In normal, non-stretched hair fibers this pattern is called an “alpha-pattern”. The alpha-pattern or α-structure of hair is characterized by specific repeated spacings (9.8 Å, 5.1 Å, and 1.5 Å). All proteins that display this X-ray diffraction pattern are called α-proteins and include, among others, human hair and nails, wool, and porcupine quill. When the hair fiber is stretched in water, a new X-ray diffraction pattern emerges that is called a “β-pattern”, with new spacings (9.8 Å, 4.65 Å, and 3.3 Å).

Damage to hair may occur in the cuticle and/or the cortex. When normal hair is damaged by heat, chemical treatment, UV radiation, and/or physical/mechanical means, myriad chemical and physical changes are induced in the hair. For example, these damaging processes have been known to produce removal or damage to cuticle scales or to cleave the thioester linkage holding the hydrophobic 18-methyl eicosanoic acid (“18-MEA”) layer to hair. Thus, it is commonly observed that undamaged hair exhibits significant hydrophobic character, whereas damaged hair shows significant hydrophilic character due to the removal of surface lipids.

There is a need, therefore, for cosmetic products that are useful in protecting the chemical and physical structure of keratinous fibers from harsh extrinsic conditions and restoring the hair's physical properties to undamaged states following damage by extrinsic conditions. More particularly, there is a need to find materials or compositions or methods that can provide a water-resistant and/or hydrophobic and/or protective barrier to hair to protect it at the cortex. Such a protective barrier should not be easily transferred from the substrate over time by normal everyday activity. In addition, the protective barrier should be shampoo, wash or water-resistant so that the barrier is not easily removed. Non-transfer and shampoo, wash or water-resistant cosmetic, hair and skin care compositions are sought which have the advantage of forming a deposit which does not undergo even partial transfer to the substrates with which they are brought into contact (for example, clothing). It is also desirable to have compositions that do not easily “run off” or wash off the skin and lips when exposed to water, rain or tears. Accordingly, a product that provides a protective barrier to the substrate that also is shampoo, wash or water resistant and non-transferable would be of benefit to the area of cosmetic products. As such, makers of cosmetic products such as hair and skin care products continue to seek materials and ingredients that can provide such benefits.

In addition, in today's market, many consumers prefer the flexibility of non-permanent hairstyles, that is, those styles obtained via non-permanent shaping of the hair. Typically, such non-permanent styles disappear when the hair is wetted, especially when the hair is washed with water and/or shampoo or when the hair is exposed to high humidity conditions. Methods for non-permanent shaping of keratinous fibers include, for example, brushing, teasing, braiding, the use of hair rollers, and heat styling, optionally with a commercially available styling product. Non-limiting examples of heat styling include blow drying, crimping, curling, and straightening methods using elevated temperatures (such as, for example, setting hair in curlers and heating, and curling with a curling iron and/or hot/steam rollers and/or flat iron).

While such compositions and methods may provide for non-permanent shaping of keratinous fibers, many consumers also desire longer lasting or durable styling/shaping than most known materials (e.g., film-forming agents, resins, gums, and/or adhesive polymers), commercially available products (e.g., conventional hair sprays, mousses, gels and lotions), and methods employing these materials and products provide. For example, many consumers desire compositions and methods that improve and preserve non-permanent curl formation or hair style.

Further, many people desire compositions and methods for retaining a particular non-permanent shape or style of keratinous fibers such as hair. A common way to retain a particular hairstyle is with the use of a hairspray, typically applied after styling the hair. Other methods to retain a hairstyle or shape of keratinous fibers include the use of mousses, gels, and lotions. The materials in these compositions are generally film forming agents, resins, gums, and/or adhesive polymers.

There is a need, therefore, for materials, compositions and methods that result in more durable or longer lasting shape or style even when the styled/shaped/curled hair is exposed to adverse environmental and physical factors and/or when wetted, washed, or shampooed.

To achieve at least one of these and other advantages, the present invention provides a method of protecting and/or repairing a keratinous substrate, and more particularly, a keratinous fiber chosen from hair, eyelashes and eyebrows from extrinsic damage caused by heating, UV radiation or chemical treatment by applying to said keratinous fiber a composition that includes a polycarbodiimide compound and a carboxylic acid compound chosen from fatty acids, their salts, and mixtures thereof in an amount effective to confer or improve the keratinous fiber's hydrophobicity.

### BRIEF SUMMARY OF THE INVENTION

In an exemplary embodiment, a keratinous treatment composition including a polycarbodiimide compound and a carboxylic acid compound chosen from fatty acids, their salts, and mixtures thereof is disclosed. The composition includes about 0.25 to about 10.0%, or about 0.5 to about 8%, or about 1 to about 5% by weight, based on the total weight of the composition, of a combined amount of the polycarbodiimide compound and the carboxylic acid compound chosen from fatty acids, their salts, and mixtures thereof. The composition includes amounts of each of the polycarbodiimide compound and the carboxylic acid compound chosen from fatty acids, their salts, and mixtures thereof sufficient to impart one or more of hydrophobicity or increased hydrophobicity to the keratinous substrate after application thereto and increased hold to the shape or configuration of the keratinous.

In another exemplary embodiment, a method of protecting a keratinous substrate chosen from hair, eyelashes and eyebrows from extrinsic damage caused by heating, UV radiation or chemical treatment, or of repairing a keratinous substrate chosen from hair, eyelashes and eyebrows following extrinsic damage caused by heating, UV radiation or chemical treatment. The method includes applying to the keratinous substrate a composition including the polycarbodiimide and the carboxylic acid compound chosen from fatty acids, their salts, and mixtures thereof in an amount effective to protect or repair the keratinous substrate; wherein the polycarbodiimide compound is present at a concentration of from about 0.15% to about 8% by weight, based on the total weight of the composition.

In another exemplary embodiment, a keratinous treatment composition including a polycarbodiimide compound and a polycarboxylic acid compound is disclosed. The composition includes from about 0.1% to about 20.0% by weight, based on the total weight of the composition, of a combined amount of the polycarbodiimide compound and the polycarboxylic acid. The composition includes amounts of each of the polycarbodiimide compound and the polycarboxylic acid compound sufficient to impart hydrophobicity to or improve the hydrophobicity of keratinous substrates such as keratinous fibers or hair, upon application thereto.

In another exemplary embodiment, a method of protecting a keratinous fiber chosen from hair, eyelashes and eyebrows from extrinsic damage caused by heating, UV radiation or chemical treatment, or of repairing a keratinous fiber chosen from hair, eyelashes and eyebrows following extrinsic damage caused by heating, UV radiation or chemical treatment. The method includes applying to the keratinous fiber a composition including the polycarbodiimide and polycarboxylic acid compound in an amount effective to protect or repair the keratinous fiber; wherein the polycarbodiimide compound is present at a concentration of from about 0.05% to about 18.0% by weight, based on the total weight of the composition.

In another exemplary embodiment, a method for durable non-permanent shaping of at least one keratinous fiber or for durable retention of a non-permanent shape of at least one keratinous fiber is disclosed. The method includes applying to the at least one keratinous fiber the composition including the polycarbodiimide and polycarboxylic acid compound.

In yet another exemplary embodiment, a keratinous treatment composition including a polycarbodiimide compound and a cationic polymer is disclosed. The composition includes from about 0.5% to about 40.0% by weight, and in particular embodiments from about 2% to about 4%, based on the total weight of the composition, of a combined amount of the polycarbodiimide compound and the cationic polymer. The composition includes amounts of each of the polycarbodiimide compound and the cationic polymer sufficient to impart hydrophobicity to or improve the hydrophobicity of keratinous substrates such as keratinous fibers or hair, upon application thereto.

In another exemplary embodiment, a method of protecting a keratinous fiber chosen from hair, eyelashes and eyebrows from extrinsic damage caused by heating, UV radiation or chemical treatment, or of repairing a keratinous fiber chosen from hair, eyelashes and eyebrows following extrinsic damage caused by heating, UV radiation or chemical treatment. The method includes applying to the keratinous fiber a composition including the polycarbodiimide and cationic polymer in an amount effective to protect or repair the keratinous fiber; wherein the polycarbodiimide compound is present at a concentration of from 0.25 to 20.0% by weight, based on the total weight of the composition.

Another embodiment of the present invention is method protecting a keratinous substrate chosen from hair comprising applying to the keratinous fiber the composition of the present invention in an amount effective to protect or repair said keratinous substrate before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

In some embodiments, the keratinous substrate in any one of the above described compositions is heated and the composition is applied to the substrate prior to heating or during heating or after heating the substrate.

In another exemplary embodiment, a method for durable non-permanent shaping of at least one keratinous substrate or for durable retention of a non-permanent shape of at least one keratinous substrate is disclosed. The method includes applying to said at least one keratinous substrate the composition including the polycarbodiimide compound and at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer.

In some embodiments, the above-described method includes a step of heating the keratinous substrate prior to or during or after the application of any of the above-described compositions.

In another exemplary embodiment, a method for durable non-permanent shaping of at least one keratinous fiber or for durable retention of a non-permanent shape of at least one keratinous fiber is disclosed. The method includes applying a composition as provided above to the at least one keratinous fiber.

According to some such embodiments, the method includes a one-step process, wherein a composition including the polycarbodiimide and the at least one additional active, and solvent is prepared and applied onto the keratinous substrate.

According to other embodiments, the method includes a two-step application process, wherein individual components (polycarbodiimide combined with a solvent and the at least one additional active combined with a solvent) are applied to the keratinous substrate in a step-wise fashion in any order to treat the substrate. In one such embodiment, the keratinous substrate is first treated with the polycarbodiimide phase, followed by treatment with the phase containing the at least one additional active.

In some embodiments, the above-described method includes a step of heating the keratinous fiber prior to or during or after the application of the above-described composition.

The present invention is also directed to a method for cosmetic treatment of keratinous tissues, such as keratinous fibers, by applying any one of the above-disclosed compositions onto a surface of the keratinous tissue, such as the cuticle of hair fibers.

The present invention is also directed to methods and kits for cosmetic treatment of keratinous tissues, such as keratinous fibers, by applying any one of the above-disclosed compositions onto a surface of the keratinous tissue, such as the cuticle of hair fibers in a step wise fashion. According to some such embodiments, the kit includes separate packaging of one or more of the actives of the inventive composition provided in one or more of thickened or un-thickened aqueous and non-aqueous phases, and packaging of any of the foregoing with one or more of processing agents selected from a coloring agent, a pigmenting agent, a permanent process agent, a relaxing process agent, a straightening process agent, and a highlighting process agent.

Other features and advantages of the present invention will be apparent from the following more detailed description of the representative embodiment which illustrates, by way of example, the principles of the invention.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“Active material” as used herein with respect to the percent amount of an ingredient or raw material, refers to 100% activity of the ingredient or raw material.

As used herein, the terms “applying a composition onto keratin fibers” and “applying a composition onto hair” and variations of these phrases are intended to mean contacting the fibers or hair, with at least one of the compositions of the invention, in any manner.

“At least one,” as used herein, means one or more and thus includes individual components as well as mixtures/combinations.

The term “comprising” (and its grammatical variations) as used herein is used in the inclusive sense of “having” or “including” and not in the exclusive sense of “consisting only of.”

“Conditioning,” as used herein, means imparting at least one of combability, manageability, moisture-retentivity, luster, shine, softness, and body to the hair.

“Durable conditioning,” as used herein, means that, following at least one shampoo/washing/rinsing after treatment of keratinous fibers such as hair with the compositions of the present disclosure, treated hair still remains in a more conditioned state as compared to untreated hair. The state of conditioning can be evaluated by measuring, and comparing, the ease of combability of the treated hair and of the untreated hair in terms of combing work (gm-in) and/or the substantivity of the conditioning agent on the hair and/or the hydrophobicity of hair which can be assessed by contact angle measurements (spread of a water droplet on the surface of the hair).

“Durable retention of a shape,” as used herein, means that, following at least one shampoo/washing/rinsing after treatment of keratinous fibers such as hair with the compositions of the present disclosure, treated hair still exhibits the ability to retain a particular or desirable shape after styling as compared to the exhibited ability of untreated hair to retain a particular or desirable shape after styling. “Durable retention of a shape” can also be related to the hydrophobicity of hair which can be assessed by contact angle measurements (spread of a water droplet on the surface of the hair).

“Durable shaping,” as used herein, refers to holding or keeping a shape of a keratinous fiber until the keratinous fiber is washed with water and/or shampoo. Retention of a shape can be evaluated by measuring, and comparing, the ability to retain a curl under conditions of high relative humidity of the treated hair and of the untreated hair in terms of Curl Efficiency. “Durable shaping” can also be related to imparting hydrophobicity to hair which can be assessed by contact angle measurements (spread of a water droplet on the surface of the hair).

“Heating” refers to the use of elevated temperature (i.e., above room temperature such as above 40° C.). In one embodiment, the heating in the inventive method may be provided by directly contacting the at least one keratinous fiber with a heat source, e.g., by heat styling of the at least one keratinous fiber. Non-limiting examples of heat styling by direct contact with the at least one keratinous fiber include flat ironing and curling methods using elevated temperatures (such as, for example, setting hair in curlers and heating, and curling with a curling iron and/or hot rollers). In another embodiment, the heating in the inventive method may be provided by heating the at least one keratinous fiber with a heat source which may not directly contact the at least one keratinous fiber. Non-limiting examples of heat sources which may not directly contact the at least one keratinous fiber include blow dryers, hood dryers, heating caps and steamers.

“A heat-activated” composition, as used herein, refers to a composition which, for example, shapes the at least one keratinous fiber better than the same composition which is not heated during or after application of the composition. Another example includes a composition which retains a shape of at least one keratinous fiber better than the same composition which is not heated during or after application.

“High humidity,” as defined herein, refers to atmospheric humidity above 40%.

The term “wash cycle” as used herein, refers to a step or process of washing a keratinous substrate and may include treating the substrate with a surfactant-based product (e.g., shampoo or conditioner or body wash) then washing or rinsing the substrate with water. The term “wash cycle” may also include washing or rinsing the substrate with water.

“Homogeneous” means having the visual appearance of being substantially uniform throughout, i.e., visually appears as a single-phase emulsion and/or dispersion.

“Keratinous substrate,” as used herein, includes, but is not limited to, skin, hair, and nails. “Keratinous substrate” as used herein also includes “keratinous tissue” or “keratinous fibers,” which as defined herein, may be human keratinous fibers, and may be chosen from, for example, hair, such as hair on the human head, or hair comprising of eyelashes or hair on the body.

The term “style” or styling” as used herein includes shaping, straightening, curling, or placing a keratin fiber such as hair, in a particular arrangement, form or configuration; or altering the curvature of a keratinous fiber or other substrate; or re-positioning a keratin fiber or other substrate to a different arrangement, form or configuration; or providing/maintaining a hold to the shape or configuration of the keratin fiber. In some embodiments, the hold to the shape of configuration of the fiber may be expressed as an improved bending force property.

As used herein, the terms “styling keratinous fibers” and variations thereof are understood to refer to any means or method of modifying the appearance of the keratinous fibers or the hair with respect to their spatial arrangement or configuration or curvature or form. When the keratinous fibers comprise hair on the human head, the term “styling keratinous fibers” or “styling hair” is also understood to include curling or waving or embossing the hair or smoothing or straightening the hair, or spiking the hair or providing/maintaining a hold to the shape or configuration of the keratin fiber.

The term “treat” (and its grammatical variations) as used herein refers to the application of the compositions of the present invention onto keratinous substrates such as keratinous fibers or hair or skin.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

It is an object of the present invention to provide materials and compositions and methods which provide both a protective barrier onto keratinous substrates such as hair which impart native/undamaged physical properties-such as hydrophobicity, ease of combing, etc.—to hair, in particular, damaged hair, as well as impart durable or long lasting physical properties mimicking natural/undamaged hair to treated hair. It is also an object of the present invention to provide materials and compositions and methods which improve the resistance of the keratinous substrate to humidity and other environmental conditions so as to preserve the retention of durable non-permanent shaping or for durable retention of a non-permanent shape of at least one keratinous fiber.

It has been surprisingly and unexpectedly discovered by the inventors that a composition containing the combination of polycarbodiimides and at least one additional active selected from one or more a carboxylic acid compound chosen from fatty acids, their salts, and mixtures thereof, a polycarboxylic acid compound other than one that is chosen from fatty acids, their salts, and mixtures thereof, and a cationic polymerfor cosmetic application, when applied to keratinous substrates such as hair, enhance the properties (hydrophobicity, adhesion, chemical resistance, water resistance etc.) and deliver superior performance to the substrate. In embodiments of this disclosure, the combination of polycarbodiimide compounds with a carboxylic acid compound chosen from fatty acids, their salts, and mixtures thereof enhance the properties of hair wherein the combination increases the conditioning effect (e.g., hydrophobicity, shine and smoothness), strengthens the hair, increases the stiffness and humidity resistance of hair and ameliorates the condition of damaged hair by improving the appearance and quality of hair (for example, smoother feel, softer feel, less frizzy, less dry, more discipline). In addition, the composition, according to the present disclosure, provides a significant reduction in combing force which indicates that hair is easier to comb and/or has less tangling. The polycarboxylic acid compounds can be solvent based and water based, as further described herein.

The compositions according to the present disclosure imparted increased percent curl retention on curled/shaped hair indicating higher resistance of the hair to high humidity and high temperature as compared to compositions with either material alone.

It was also surprisingly and unexpectedly discovered by the inventors that curled/shaped hair treated with the compositions according to the present disclosure maintained its shape (i.e., same or similar degree of curl retention) even after shampooing/washing the hair.

In addition, the composition, according to the present disclosure, may be applied in a one-step or multiple-step process such as a two-step process.

Without being bound to any one theory, the inventors of the present disclosure believe that the polycarbodiimide compound and at least one additional active selected from one or more a carboxylic acid compound chosen from fatty acids, their salts, and mixtures thereof, a polycarboxylic acid compound other than one that is chosen from fatty acids, their salts, and mixtures thereof, and a cationic polymercomprising the keratinous treatment compositions of the invention react to each other and to the keratin substrate when such compositions are applied onto keratinous substrates such as hair or skin. It is also believed that the compositions of the present disclosure provide a protective barrier useful in cosmetic applications such as hair care, hair styling, nail care, makeup, skin care, and sun care products such that the hydrophobicity of the keratinous substrates is improved or restored resulting in significantly better cosmeticities, feel and appearance, and less damaged condition of the substrates such as hair and skin.

The compositions according to the invention are compositions including polycarbodiimide and at least one additional active selected from one or more a carboxylic acid compound chosen from fatty acids, their salts, and mixtures thereof, a polycarboxylic acid compound other than one that is chosen from fatty acids, their salts, and mixtures thereof, and a cationic polymer. The composition may include other suitable ingredients for hair treatment or hair repair. For example, known solvents and/or additives may be utilized in addition to the polycarbodiimide compound and carboxylic acid compound chosen from fatty acids, their salts, and mixtures thereof to provide additional benefits to the composition. When both polycarbodiimide compound and carboxylic acid compound chosen from fatty acids, their salts, and mixtures thereof are utilized in combination to form the inventive composition, a significant increase in hydrophobicity of keratinous fiber is provided.

The range of concentrations by weight of the composition over which the association provides hydrophobicity to and/or hold to the shape or configuration of keratinous substrates is about 0.1% to about 10% of combined amount of the polycarbodiimide compound and carboxylic acid compound chosen from fatty acids, their salts, and mixtures thereof. The range of the weight ratio of the polycarbodiimide to the carboxylic acid compound chosen from fatty acids, their salts, and mixtures thereof in the composition over which their association provides hydrophobicity to keratinous substrates such as hair, is from about 20:1 to about 20:1, or such as from about 10:1 to about 1:10 or such as from about 20:1 to about 1:1, including all ranges and subranges there-between. In certain embodiments, the weight ratio of polycarbodiimide to carboxylic acid compound chosen from fatty acids, their salts, and mixtures thereof in the compositions of the present disclosure at which hydrophobicity is imparted to keratinous substrates is at about 1:1 or about 1:2 or about 1:3 or about 1:4 or about 1:5 or about 1:7 or about 1:7 or about 1:8 or about 1:9 or about 1:10 or about 10:1 or about 9:1 or about 8:1 or about 7:1 or about 8:1 or about 7:1 or about 6:1 or about 5:1 or about 4:1 or about 3:1 or about 2:1.

Further, the range of concentrations by weight of the composition over which the association provides hydrophobicity to and/or hold to the shape or configuration of keratinous substrates is about 0.1% to about 20% of combined amount of the polycarbodiimide compound and polycarboxylic acid compound. The range of the weight ratio of the polycarbodiimide to the polycarboxylic acid compound in the composition over which their association provides hydrophobicity to keratinous substrates such as hair, is from about 1:1 to about 1:20, or such as from about 1:1 to about 1:10 including all ranges and subranges there-between. In certain embodiments, the weight ratio of polycarbodiimide to polycarboxylic acid in the compositions of the present disclosure at which hydrophobicity is imparted to keratinous substrates is at about 1:1 or about 1:2 or about 1:3 or about 1:4 or about 1:5 or about 1:7 or about 1:7 or about 1:8 or about 1:9 or about 1:10.

Further, in accordance with other embodiments, the compositions according to the invention, are compositions including polycarbodiimide and cationic polymer compounds. The range of concentrations by weight of the composition over which the association provides hydrophobicity is 0.5% to 40% total actives. A range of ratios polycarbodiimide to cationic polymer compound of about 1:10 to about 10:1 or about 1:5 to about 5:1, including all ranges and subranges there-between, or such as about 1:1 or about 1:2 or about 1:3 or about 1:4 or about 1:5 or about 1:7 or about 1:7 or about 1:8 or about 1:9 or about 1:10 or about 10:1 or about 9:1 or about 8:1 or about 7:1 or about 8:1 or about 7:1 or about 6:1 or about 5:1 or about 4:1 or about 3:1 or about 2:1.

Actives

Polycarbodiimides

A carbodiimide group is a linear triatomic moiety generally depicted by Formula (I):

*—(N═C═N)—* (I)

At least one of the nitrogens is linked to or incorporated into a backbone or other bridging group to result in a molecule having at least two carbodiimide groups.

Polycarbodiimides

In one embodiment, the polycarbodiimides comprising of at least two carbodiimide units, as described above, can be represented by Formula (II):

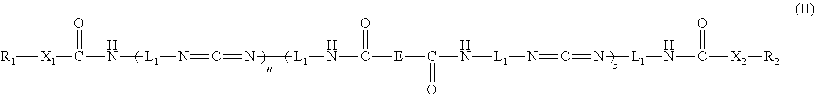

wherein X₁ and X₂ each independently represent O, S or NH. R₁ and R₂ are selected from a hydrocarbon group containing one or more catenary or non-catenary hetero-atoms, such as nitrogen, sulfur and oxygen, and linear or branched and cyclic or acyclic groups which can be ionic or non-ionic segments, or a partially or fully fluorinated hydrocarbon group that may contain one or more catenary or non-catenary hetero-atoms; n and z are, each independently, an integer of 0 to 20; L₁ (Linker of carbodiimide groups) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group, and a C₃ to C₁₂ divalent heterocyclic group; wherein a plurality of L₁s may be identical to or different from one another, and wherein in another embodiment, L₁ of formula (II) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group that is not chosen from m-tetramethylxylylene, and a C₃ to C₁₂ divalent heterocyclic group; wherein a plurality of L₁s may be identical to or different from one another;
wherein E is a radical selected from the following formulas:

O—R₃—O;S—R₄—S; and

R₅—N—R₄—N—R₅;

wherein R₃ and R₄ are each independently hydrocarbon radicals that may contain halogen atoms or one or more catenary (i.e.; in chain, bonded only to carbon) or non-catenary hetero atoms, including an aromatic, cycloaliphatic, aryl and alkyl radical (linear or branched) and R₅ is hydrogen, or a hydrocarbon radical which can contain halogen atoms or one or more catenary (i.e.; in chain, bonded only to carbon) or non-catenary hetero atoms.

Examples of R1 and R2 can be methyl glycolate, methyl lactate, polypropylene glycol, polyethylene glycol monomethyl ether, dialkylamino alcohol.

Examples of L1 can be the diradical of tolylene, hexamethylene, hydrogenated xylylene, xylylene, 2,2,4-trimethylhexamethylene, 1,12-dodecane, norbornane, 2,4-bis-(8-octyl)-1,3-dioctylcyclobutane, 4,4′-dicyclohexylmethane, tetramethylxylylene, isophorone, 1,5-naphthylene, 4,4′ diphenylmethane, 4,4′ diphenyldimethylmethane, phenylene.

Polycarbodiimides may include polymers with a plurality of carbodiimide groups appended to the polymer backbone. For example, U.S. Pat. No. 5,352,400 (the disclosure of which is incorporated by reference herein for all purposes as if fully set forth) discloses polymers and co-polymers derived from alpha-methylstyryl-isocyanates. Such a polymer is illustrated in Formula (III).

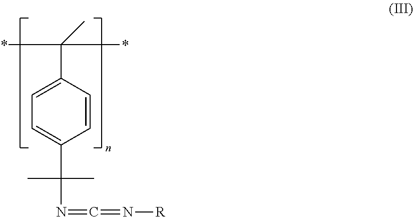

wherein R is an alkyl, cycloalkyl or aryl group (in some particular embodiments having from 1 to 24 carbon atoms).

In another embodiment, polycarbodiimides, according to the present disclosure, include polycarbodiimides having branched structures, like that shown in Formula (IV), and as described in Chapter 8 of Technology for Waterborne Coatings, E. J. Glass Ed., ACS Symposium 663, 1997; The Application of Carbodiimide Chemistry to Coating, by J. W. Taylor and D. R. Bassett (the disclosure of which is incorporated by reference herein for all purposes as if fully set forth).

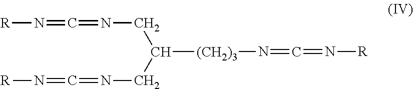

wherein R is an alkyl, cycloalkyl or aryl group (in some particular embodiments having from 1 to 24 carbon atoms).

In one embodiment, the compositions of the present disclosure does not employ a polycarbodiimide having a linker L₁ chosen from m-tetramethylxylylene.

Suitable polycarbodiimide compounds include, but are not limited to, those commercially sold by the suppliers Nisshinbo, Picassian, and 3M. Particularly suitable polycarbodiimide compounds include, but are not limited to, those known by the name under the CARBODILITE series, V-02, V02-L2, SV-02, E-02, V-10, SW-12G, E-03A, commercially sold by Nisshinbo.

In some embodiments, the polycarbodiimide of the present disclosure is selected from compounds of formula (II) wherein L1 (Linker of carbodiimide groups) represents a C1 to C18 divalent aliphatic hydrocarbon group, a C3 to C13 divalent alicyclic hydrocarbon group, a C3 to C12 divalent heterocyclic group, or a C6 to C14 divalent aromatic hydrocarbon group; wherein a plurality of L₁s may be identical to or different from one another.

In other embodiments, the polycarbodiimide of the present disclosure is selected from compounds of formula (II) wherein L1 is not chosen from m-tetramethylxylylene.

In certain embodiments, the composition of the present disclosure is devoid of a polycarbodiimide that has a linker L1 chosen from m-tetramethylxylylene.

In accordance with the various embodiments wherein the polycarbodiimide is combined with carboxylic acid compounds chosen from fatty acids, their salts, and mixtures thereof, the polycarbodiimide compound is present in the compositions according to the disclosure in amounts range from about 0.01% to about 10%, and in some embodiments from about 0.15% to about 8%, and in some embodiments from about 0.2% to about 7%, and in some further embodiments from about 0.25% to about 5%. In some representative embodiments, the polycarbodiimide compound is present from about 0.1% to about 5%, and from about 0.2% to about 3%. In yet other embodiments, the polycarbodiimide compound is present from at least 0.15%. And in yet other embodiments, the polycarbodiimide compound is present from at least 0.25% to up to about 10%. In some specific embodiments, the polycarbodiimide compound is present at about 3%, or about 2.75%, or about 2.5% or about 2%. In some specific embodiments, the polycarbodiimide compound is present at about 0.75%, or about 0.5%. In some other specific embodiments, the polycarbodiimide compound is present at about 0.25%.

Thus, the polycarbodiimide compound may be present from about 0.01, 0.05, 0.1, 0.2, 0.3, 0.4, 0.5, 0.6, 0.7, 0.8, 0.9, 1, 2, 3, 4, 5, 6, 7, 8, 9, to about 10 percent by weight, including increments and ranges therein and there between.

In accordance with the other embodiments wherein the polycarbodiimide is combined with a polycarboxylic acid compound, the polycarbodiimide compound is present in the compositions according to the disclosure in amounts range from about 0.01% to about 20%, and in some embodiments from about 0.25% to about 18%, and in some further embodiments from about 0.25% to about 10%. In some representative embodiments, the polycarbodiimide compound is present from about 0.1% to about 5%. In yet other embodiments, the polycarbodiimide compound is present from at least 0.1%. And in yet other embodiments, the polycarbodiimide compound is present from at least 0.1% to up to about 18%. In some specific embodiments, the polycarbodiimide compound is present at about 2.5%, or about 3%, or about 4%, or about 5%, or about 6%. In some specific embodiments, the polycarbodiimide compound is present at about 2% to about 4% In some other specific embodiments, the polycarbodiimide compound is present at about 5% to about 8%.

Thus, the polycarbodiimide compound may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and therebetween.

In accordance with yet other embodiments wherein the polycarbodiimide is combined with a cationic polymer, the polycarbodiimide compound is present in the compositions according to the disclosure in amounts range from about 0.01% to about 20%, and in some embodiments from about 0.01% to about 15%, and in some further embodiments from about 0.5% to about 10%. In some representative embodiments, the polycarbodiimide compound is present from about 0.5% to about 5%. In yet other embodiments, the polycarbodiimide compound is present from at least 0.5%. And in yet other embodiments, the polycarbodiimide compound is present from at least 0.5% to up to about 20%. In some specific embodiments, the polycarbodiimide compound is present at about 2%, or about 3%, or about 4%, or about 5%, or about 6%. In some specific embodiments, the polycarbodiimide compound is present at about 3% to about 4% In some other specific embodiments, the polycarbodiimide compound is present at about 6% to about 8%.

Thus, the polycarbodiimide compound may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and therebetween.

Carboxylic Acid Compound Chosen from Fatty Acids

The compositions of the present disclosure employ at least one carboxylic acid compound chosen from fatty acids, their salts, and mixtures thereof.

The carboxylic acid compound of the present disclosure may generally be chosen from saturated or unsaturated carboxylic acids having carbon chains containing from 6 to 30 carbon atoms, in certain embodiments from 9 to 30 carbon atoms, and in certain embodiments from 9 to 22 carbon atoms and wherein the carbon chain is optionally substituted, for example with one or more (in particular 1 to 4) hydroxyl groups. If the fatty acids of the present disclosure are unsaturated, these compounds may comprise one to three conjugated or unconjugated carbon-carbon double bonds.

Suitable examples of the carboxylic acid compound of the present disclosure are oleic acid, linoleic acid, linolenic acid, isostearic acid, caproic acid, capric acid, caprylic acid, oleic acid, linoleic acid, linolenic acid, behenic acid, lauric acid, myristic acid, stearic acid, palmitic acid, hydroxystearic acid, dilinoleic acid, octadecenedioic acid, and mixtures thereof.

The carboxylic acid compound of the present disclosure may also be chosen from salts of fatty acids, in particular, alkali metal salts of fatty acids (metal soaps) and organic base salts of fatty acids.

The alkali metals that may be used include sodium, potassium, lithium and their mixtures.

In certain embodiments, the alkali metal is sodium.

In some embodiments, the metal of the alkali metal salts of fatty acids can in particular be sodium, zinc or magnesium.

In other embodiments, the organic base salts of fatty acids may be obtained from the neutralization of fatty acids with organic bases such as ammonia, monoethanolamine or triethanolamine.

The neutralization rate of fatty acid may range from 70 to 90 percent, most in certain embodiments in the range from 80 to 85 percent.

The alkali metal salts of fatty acids and organic base salts of fatty acids that may be used in the present disclosure include sodium stearate, zinc laurate, magnesium stearate, magnesium myristate, zinc stearate, ammonium stearate, ammonium oleate, ammonium nonanoate, and their mixtures.

Another example of the alkali metal of a fatty acid is potassium cocoate, also known as potassium coco soap that originates from the fatty acids present in coconut oil.

In certain embodiments, the carboxylic acid compounds of the present disclosure are in certain embodiments chosen from oleic acid, linoleic acid, linolenic acid, isostearic acid, hydroxystearic acid, dilinoleic acid, octadecenedioic acid, and mixtures thereof.

In other embodiments, the carboxylic acid compound which is suitable for the present disclosure is chosen from mixtures of C10-C30 fatty acids of plant or animal origin such as the mixture contained in coconut oil, also known as coprah oil or copra oil.

In some embodiments, the carboxylic acid compound of the present disclosure are not in the form of salts, i.e. if present, the composition may not contain organic or mineral alkaline agents such as sodium hydroxide, potassium hydroxide, monoethanolamine, triethanolamine, ammonia.

In accordance with the various embodiments wherein the polycarbodiimide is combined with carboxylic acid compounds chosen from fatty acids, their salts, and mixtures thereof, the carboxylic acid compound is present in the compositions according to the disclosure in amounts range from about 0.01% to about 10%, and in some embodiments from about 0.15% to about 8%, and in some embodiments from about 0.2% to about 7%, and in some further embodiments from about 0.25% to about 5%. In some representative embodiments, the carboxylic acid compound is present from about 0.1% to about 5%, and from about 0.2% to about 3%. In yet other embodiments, the carboxylic acid compound is present from at least 0.15%. And in yet other embodiments, the polycarbodiimide compound is present from at least 0.25% to up to about 10%. In some specific embodiments, the carboxylic acid compound is present at about 3%, or about 2.75%, or about 2.5% or about 2%. In some specific embodiments, the carboxylic acid compound is present at about 0.75%, or about 0.5%. In some other specific embodiments, the carboxylic acid compound is present at about 0.25%.

Thus, the carboxylic acid compound may be present from about 0.01, 0.05, 0.1, 0.2, 0.3, 0.4, 0.5, 0.6, 0.7, 0.8, 0.9, 1, 2, 3, 4, 5, 6, 7, 8, 9, to about 10 percent by weight, including increments and ranges therein and there between.

Polycarboxylic Acid Polymer Compounds (other than the carboxylic acid compound chosen from fatty acids, their salts, and mixtures thereof of the present invention)

The polycarboxylic acid compounds of the present disclosure may be chosen from anionic, nonionic, and amphoteric polymers.

The anionic polymers may be soluble in a cosmetically acceptable medium or insoluble in this same medium such that they may be used in the form of dispersions of solid or liquid particles of polymer (latex or pseudolatex).

The anionic polymers may be selected from polymers comprising groups derived from carboxylic acids, and have an average molecular weight by number of between about 500 and 5,000,000. The carboxylic groups are provided by unsaturated mono- or diacid carboxylic monomers such as those that have the formula:

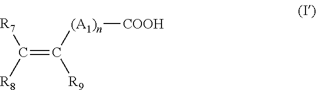

in which n is an integer from 0 to 10, A₁ denotes a methylene group optionally joined to the carbon atom of the unsaturated group or to the adjacent methylene group when n is greater than 1, via a heteroatom such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group, a CH₂—COOH, phenyl or benzyl group.

In the abovementioned formula, a lower alkyl group in certain embodiments denotes a group containing 1 to 4 carbon atoms and in particular methyl and ethyl groups.

The polycarboxylic acid compounds include Copolymers of acrylic or methacrylic acid or salts thereof, and in particular copolymers of acrylic acid and acrylamide sold in the form of their sodium salts;

The polycarboxylic acid compounds include Copolymers of acrylic or methacrylic acid with a monoethylenic monomer such as ethylene, styrene, vinyl esters and acrylic or methacrylic acid esters, optionally grafted onto a polyalkylene glycol such as polyethylene glycol and optionally crosslinked. The polycarboxylic acid compounds include methacrylic acid/acrylic acid/ethyl acrylate/methyl methacrylate copolymers in an aqueous dispersion.

The polycarboxylic acid compounds include Crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an [alpha]- or [beta]-cyclic carboxylic acid.

The polycarboxylic acid compounds include Copolymers of C4-C8 monounsaturated carboxylic acids selected from: copolymers comprising (i) one or more maleic, fumaric, itaconic, allyloxyacetic, methallyloxyacetic, 3-allyloxypropionic, allylthioacetic, allylaminoacetic, vinylacetic, vinyloxyacetic, crotyloxyacetic, 3-butenoic, 4-pentenoic, 10-undecenoic, allylmalonic, maleamic, itaconamic, N-monohydroxyalkyl- or N-dihydroxy-alkyl-maleamic acids and (ii) at least one monomer selected from vinyl esters, vinyl ethers, vinyl halides, phenylvinyl derivatives, acrylic acid and its esters, the anhydride functions of these copolymers optionally being monoesterified or monoamidated. The polycarboxylic acid compounds include copolymers comprising (i) one or more maleic, citraconic or itaconic anhydride units and (ii) one or more monomers selected from allyl or methallyl esters optionally comprising one or more acrylamide, methacrylamide, [alpha]-olefin, acrylic or methacrylic ester, acrylic or methacrylic acid or vinylpyrrolidone groups in their chain, the anhydride functions of these copolymers optionally being monoesterified or monoamidated. The polycarboxylic acid compounds include polyacrylamides comprising carboxylate groups.

The polycarboxylic acid compounds of the present disclosure may also include those anionic polymers as sold under the FIXATE series as commercially available from Lubrizol, such as a branched block anionic polymer sold as FIXATE G-100, a branched anionic acrylate copolymer Polyacrylate-2 Crosspolymer (FIXATE SUPERHOLD polymer), Acrylates Crosspolymer-3 (FIXATE FREESTYLE Polymer), Polyacrylate-14 (FIXATE PLUS Polymer), those sold under the CARBOPOL series as commercially available from Lubrizol such as Acrylates Crosspolymer-4 (CARBOPOL AQUA SF-2), Acrylates Crosspolymer-4 (CARBOPOL AQUA CC), and those sold under the SYNTRAN series as commercially available from Interpolymer such as Acrylates Copolymer (SYNTRAN 5190), Styrene/Acrylates/Ammonium Methacrylate Copolymer (SYNTRAN 5760), and Ammonium Acrylates Copolymer (SYNTRAN KL-219C).

The polycarboxylic acid compounds of the present disclosure also includes anionic latex polymers such as acrylic copolymer and (meth)acrylate copolymers dispersions.

The polycarboxylic acid compounds include copolymers of acrylic acid or of acrylic esters, such as Acrylates/t-Butylacrylamide copolymer sold as ULTRAHOLD 8, acrylic acid/ethyl acrylate/N-tert-butylacrylamide terpolymers sold especially as ULTRAHOLD STRONG by BASF, copolymers derived from crotonic acid, such as vinyl acetate/vinyl tert-butylbenzoate/crotonic acid terpolymers and the crotonic acid/vinyl acetate/vinyl neododecanoate terpolymers sold especially as RESYN 28-29-30 by Azko Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, as GANTREZ AN or ES by ISP, the copolymers of methacrylic acid and methyl methacrylate sold as EUDRAGIT L by Rohm Pharma, the copolymers of methacrylic acid and ethyl acrylate sold as LUVIMER MAEX or MAE by BASF, the vinyl acetate/crotonic acid copolymers sold as LUVISET CA 66 by BASF, the vinyl acetate/crotonic acid copolymers grafted with polyethylene glycol sold as ARISTOFLEX A by BASF, the polymer sold as FIXATE G-100 by Noveon, and an alpha olefin hydrocarbon-maleic anhydride copolymer wax commercially available from Clariant under the tradename LICOCARE CM 401 LP 3345 (or “Clariant CM 401”).

The polycarboxylic acid compounds include amphoteric polymers which may be selected from the following polymers: copolymers having acidic vinyl units and basic vinyl units, such as those resulting from the copolymerization of a monomer derived from a vinyl compound bearing a carboxylic group such as, more particularly, acrylic acid, methacrylic acid, maleic acid, alpha-chloroacrylic acid, and a basic monomer derived from a substituted vinyl compound containing at least one basic atom, such as, more particularly, dialkylaminoalkyl methacrylate and acrylate, dialkylaminoalkylmethacrylamides and acrylamides. Such compounds are described in U.S. Pat. No. 3,836,537.

The polycarboxylic acid compounds include Polymers comprising units derived from:

at least one monomer selected from acrylamides and methacrylamides substituted on the nitrogen atom with an alkyl group,

at least one acidic comonomer containing one or more reactive carboxylic groups, selected more particularly from acrylic, methacrylic, crotonic, itaconic, maleic and fumaric acid and alkyl monoesters, having 1 to 4 carbon atoms, of maleic or fumaric acid or anhydride, and

at least one basic comonomer such as esters with primary, secondary, tertiary or quaternary amine substituents of acrylic and methacrylic acids and the product of quaternization of dimethylaminoethyl methacrylate with dimethyl or diethyl sulfate. The N-substituted acrylamides or methacrylamides that are more particularly representative according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides. The representative basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold as AMPHOMER LV 71, Acrylates/octylacrylamide copolymer sold as AMPHOMER 28-4961 or LOVOCRYL 47 by National Starch, are particularly used.

The polycarboxylic acid compounds include Crosslinked and acylated polyaminoamides.

The polycarboxylic acid compounds include carboxylic acids selected from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The polycarboxylic acid compounds include Polymers comprising zwitterionic units of formula:

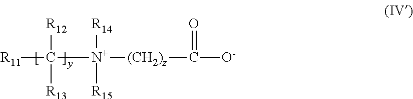

in which R11 denotes a polymerizable unsaturated group such as an acrylate, methacrylate, acrylamide or methacrylamide group, y and z represent an integer from 1 to 3, R12 and R13 represent a hydrogen atom, a methyl, ethyl or propyl group, R14 and R15 represent a hydrogen atom or an alkyl group such that the sum of the carbon atoms in R14 and R15 does not exceed 10.

The polymers comprising such units may also comprise units derived from non-zwitterionic monomers such as dimethyl- or diethylaminoethyl acrylate or methacrylate or alkyl acrylates or methacrylates, acrylamides or methacrylamides or vinyl acetate, for example, methyl methacrylate/methyl dimethylcarboxymethylammonioethyl methacrylate copolymers such sold as DIAFORMER Z301 by Sandoz; The polycarboxylic acid compounds include Polymers derived from chitosan comprising monomer units corresponding to the following formulae:

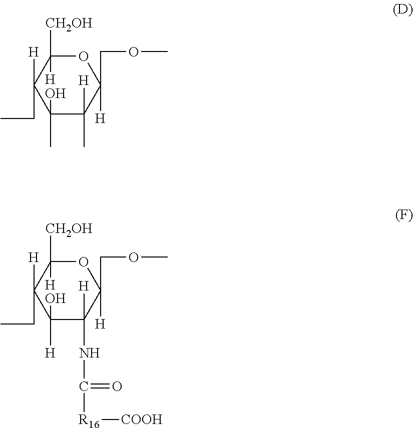

the unit (D) being present in proportions of between 0 and 30 percent, the unit (E) in proportions of between 5 percent and 50 percent and the unit (F) in proportions of between 30 percent and 90 percent, it being understood that, in this unit (F), R16 represents a group of formula:

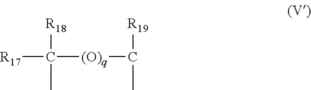

in which, if q=0, R17, R18 and R19, which may be identical or different, each represent a hydrogen atom, a methyl, hydroxyl, acetoxy or amino residue, a monoalkylamine residue or a dialkylamine residue that are optionally interspersed with one or more nitrogen atoms and/or optionally substituted with one or more amine, hydroxyl, carboxyl, alkylthio or sulfonic groups, an alkylthio residue in which the alkyl group bears an amino residue, at least one of the groups R17, R18 and R19 being, in this case, a hydrogen atom;

or, if q=1, R17, R18 and R19 each represent a hydrogen atom, and also the salts formed by these compounds with bases or acids.

The polycarboxylic acid compounds include Polymers with units corresponding to the general formula (VI′) are described, for example, in French patent 1 400 366:

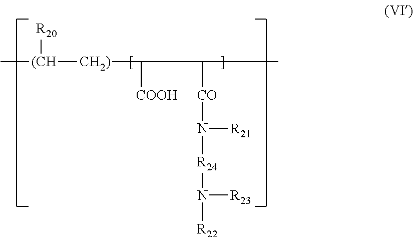

in which R20 represents a hydrogen atom, a CH3O, CH3CH2O or phenyl group, R21 denotes a hydrogen atom or a lower alkyl group such as methyl or ethyl, R22 denotes a hydrogen atom or a C1-C6 lower alkyl group such as methyl or ethyl, R23 denotes a C1-C6 lower alkyl group such as methyl or ethyl or a group corresponding to the formula: —R24-N(R22)2, R24 representing a group —CH2-CH2-, —CH2-CH2-CH2- or —CH2-CH(CH3)-, R22 having the meanings mentioned above.

The polycarboxylic acid compounds include Polymers derived from the N-carboxyalkylation of chitosan, such as N-carboxymethylchitosan or N-carboxybutylchitosan.

The polycarboxylic acid compounds include Amphoteric polymers of the type -D-X-D-X selected from:

a) Polymers obtained by the action of chloroacetic acid or sodium chloroacetate on compounds comprising at least one unit of formula:

-D-X-D-X-D- (VII′) where D denotes a

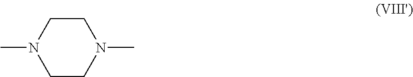

group and X denotes the symbol E or E′; E or E′ may be identical or different and denote a divalent group that is an alkylene group with a straight or branched chain containing up to 7 carbon atoms in the main chain, which is unsubstituted or substituted by hydroxyl groups and which may comprise, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) Polymers having the formula:

-D-X-D-X—(X′) where D denotes a

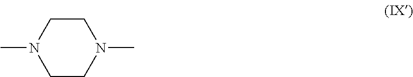

group and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interspersed by an oxygen atom and necessarily comprising one or more carboxyl functions or one or more hydroxyl functions and betainized by reaction with chloroacetic acid or sodium chloroacetate.

The polycarboxylic acid compounds include (C1-C5)Alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also comprise other vinyl comonomers such as vinylcaprolactam.

Among the amphoteric polymers described above, the ones that are most representative are Octylacrylamide/acrylates/butylamino ethyl methacrylate copolymer, such as the products sold as AMPHOMER, AMPHOMER LV 71 or LOVOCRYL 47 by National Starch and the copolymers of methyl methacrylate/methyl dimethylcarboxy-methylammonioethyl methacrylate, sold, for example, as DIAFORMER Z301 by Sandoz.

The polycarboxylic acids of the present disclosure may be chosen from compounds which are known to be used as rheology modifiers or thickeners in cosmetic compositions. Such polymers include anionic and amphoteric polymers, for example crosslinked homopolymers of acrylic acid, associative polymers, non-associative thickening polymers, and water-soluble thickening polymers. Such polymers may also be chosen from nonionic, anionic, cationic and amphoteric amphiphilic polymers. The rheology modifiers or thickeners that can used may include those polycarboxylic acid compounds described above.

The amphiphilic polymers may, optionally, contain a hydrophobic chain that is a saturated or unsaturated, aromatic or non-aromatic, linear or branched C6-C30 hydrocarbon-based chain, optionally comprising one or more oxyalkylene (oxyethylene and/or oxypropylene) units.

Representative examples of such amphiphilic polymers are:

nonionic amphiphilic polymers containing a hydrophobic chain such as:

copolymers of C1-C6 alkyl(meth)acrylates and of amphiphilic monomers containing a hydrophobic chain;

copolymers of hydrophilic (meth)acrylates and of hydrophobic monomers containing at least one hydrophobic chain, for instance the polyethylene glycol methacrylate/lauryl methacrylate copolymer;

anionic amphiphilic polymers containing at least one hydrophobic chain which are crosslinked or non-crosslinked, contain at least one hydrophilic unit derived from one or more ethylenically unsaturated monomers bearing a carboxylic acid function, which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

Examples of anionic amphiphilic polymers include CARBOPOL ETD-2020 (acrylic acid/C10-C30 alkyl methacrylate crosslinked copolymer sold by the company Noveon); CARBOPOL 1382, PEMULEN TR1 and PEMULEN TR2 (acrylic acid/C10-C30 alkyl acrylate crosslinked copolymers-sold by the company Noveon), the methacrylic acid/ethyl acrylate/oxyethylenated stearyl methacrylate copolymer (55/35/10); the (meth)acrylic acid/ethyl acrylate/25 EO oxyethylenated behenyl methacrylate copolymer (ACULYN 28 sold by Rohm and Haas) and the methacrylic acid/ethyl acrylate/steareth-10 allyl ether crosslinked copolymer.

Other examples include cross-linked acrylic polymers, for example those sold under the CARBOPOL SF series, such as ethyl acrylate/methacrylic acid copolymer with INCI name: acrylates copolymer, sold under the name CARBOPOL SF1 by the LUBRIZOL company.

Yet other examples include anionic polymers also known as anionic thickening polymers chosen from carbomers, acrylate copolymers, and crosslinked terpolymers of methacrylic acid, ethylacrylate, and polyethylene glycol (10 EO) stearyl alcohol ether (Steareth 10), such as the products sold by the company ALLIED COLLOIDS under the names SALCARE SC 80 and SALCARE SC 90, which are aqueous emulsions containing 30 percent of a crosslinked terpolymer of methacrylic acid, of ethyl acrylate and of steareth-10-allyl ether (40/50/10).

Anionic thickening polymers can also be chosen from:

terpolymers formed from maleic anhydride/C30-C38 alpha-olefin/alkyl maleate such as the product (maleic anhydride/C30-C38 alpha-olefin/isopropyl maleate copolymer) sold under the name PERFORMA 1608 by the company NEWPHASE TECHNOLOGIES;

acrylic terpolymers formed from: (a) 20 percent to 70 percent by weight of a carboxylic acid with alpha, beta-monoethylenic unsaturation; (b) 20 percent to 80 percent by weight of a nonsurfactant monomer with alpha, beta-monoethylenic unsaturation different from (a); and (c) 0.5 percent to 60 percent by weight of a nonionic monourethane which is the product of the reaction of a monohydric surfactant with a monoisocyanate with monoethylenic unsaturation; (3) copolymers formed from at least two monomers, wherein at least one of the two monomers is chosen from a carboxylic acid with alpha, beta-monoethylenic unsaturation, an ester of a carboxylic acid with alpha, beta-monoethylenic unsaturation, and an oxyalkylenated fatty alcohol; and (4) copolymers formed from at least three monomers, wherein at least one of the three monomers is chosen from a carboxylic acid with alpha, beta-monoethylenic unsaturation, at least one of the three monomers is chosen from an ester of a carboxylic acid with alpha beta-monoethylenic unsaturation and at least one of the three monomers is chosen from an oxyalkylenated fatty alcohol.

Additionally, these compounds can also contain, as a monomer, a carboxylic acid ester comprising an apha, beta-monoethylenic unsaturation and a C1-C4 alcohol. By way of example of this type of compound, there may be mentioned ACULYN 22 sold by the company ROHM and HAAS, which is an oxyalkylenated stearyl methacrylate/ethylacrylate/methacrylic acid terpolymer.

The polycarboxylic acid compounds include associative polyurethanes, associative unsaturated polyacids, and associative polymers or copolymers containing at least one monomer comprising ethylenic unsaturation.

A representative example of an associative polyurethane is methacrylic acid/methyl acrylate/ethoxylated (40 EO) behenyl alcohol dimethyl(meta-isopropenyl)benzyl isocyanate terpolymer as a 25 percent aqueous dispersion, known by the trade name, VISCOPHOBE DB 1000 and commercially available from Amerchol.

According to some embodiments of the present invention, the polycarboxylic acid compounds include at least one acrylic acid-based, (meth)acrylic acid-based, acrylate-based or (meth)acrylate-based monomer having anionic and/or cationic functionalities. Suitable compounds include, but are not limited to, polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co)polymers, or similar (co)polymers, can be combined individually or with other (co)polymers in such a way to form suitable bimodal agents having both cationic and anionic functionalities. According to certain embodiments, the bimodal agent is selected from the group consisting of polymers consisting of polyacrylate-21 and acrylates/dimethylaminoethylmethacrylate copolymer (marketed under the name SYNTRAN PC 5100 by Interpolymer), polyacrylate-16 (marketed under the name SYNTRAN PC 5112 by Interpolymer), and polyacrylate-18 and polyacrylate-19 (marketed under the names SYNTRAN PC 5107 or SYNTRAN PC 5117 by Interpolymer).

In certain embodiments, the at least one polycarboxylic acid compound of the present disclosure is selected from Octylacrylamide/acrylates/butylamino ethyl (metha)crylate copolymer, acrylic acid/C10-C30 alkyl acrylate crosslinked copolymers, crotonic acid/vinyl acetate/vinyl neododecanoate terpolymers, acrylates copolymer, polyacrylate-2, polyacrylate-21, oxyalkylenated stearyl methacrylate/ethylacrylate/methacrylic acid terpolymer, methacrylic acid/ethyl acrylate/oxyethylenated stearyl methacrylate copolymer (55/35/10); (meth)acrylic acid/ethyl acrylate/25 EO oxyethylenated behenyl methacrylate copolymer, methacrylic acid/ethyl acrylate/steareth-10 allyl ether crosslinked copolymer, an alpha olefin hydrocarbon-maleic anhydride copolymer wax and mixtures thereof.

In accordance with the other embodiments wherein the polycarbodiimide is combined with a polycarboxylic acid compound, at least one polycarboxylic acid compound is present in the compositions according to the disclosure in amounts range from about 0.01% to about 20%, and in some embodiments from about 0.01% to about 15%, and in some further embodiments from about 0.05% to about 10%. In some representative embodiments, the polycarboxylic acid compound is present from about 0.1% to about 5%. In yet other embodiments, the polycarboxylic acid compound is present from at least 0.1%. And in yet other embodiments, the polycarboxylic acid compound is present from at least 0.1% to up to about 18%. In some specific embodiments, the polycarboxylic acid compound is present at about 2.5%, or about 3%, or about 4%, or about 5%, or about 6%. In some specific embodiments, the polycarboxylic acid compound is present at about 2% to about 4% In some other specific embodiments, the polycarboxylic acid compound is present at about 5% to about 8%.

Thus, the polycarboxylic acid compound may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and therebetween.

Cationic Polymers

The cationic polymers of the present disclosure contain at least one carboxyl group.

The cationic polymer can have a negative charge but remains cationic overall, can be an amphoteric polymer that can carry a cationic charge based on pH, or can be a betaine polymer that remains amphoteric at any pH.

The cationic polymers are polymers that result from the homopolymerization or copolymerization of ethylenically unsaturated monomers chosen from: (i) at least one nonionic monomer such as (Alkyl)(Meth)Acrylamide, (Alkyl)(Meth)Acrylate Ester, Vinyl Pyrrolidone, Vinyl Imidazole; (ii) at least one cationic monomer such as Ethyltrimonium (Alkyl)(Meth)Acrylamide, Ethyltrimonium (Alkyl)(Meth)Acrylate Ester, Vinylimidazoline, Dimethylaminopropyl (Alkyl)(Meth)Acrylamide, Methacrylamidopropyl Triethyl Ammonium Chloride (MAPTAC), Diallyl Dimethyl Ammonium Chloride (DADMAC); (iii) at least one (Alkyl)Acrylic acid; (iv) at least one amphoteric monomer such as a carboxybetaine zwitterionic monomer.

Suitable examples of such cationic polymers are: the diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquatemium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acryl amide/dimethyldi allylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name: Polyquaternium-53); Polyquatemium-30, Polyquatemium-35, Polyquaternium-45, Polyquatemium-50, Polyquaternium-54; Polyquaternium-57; Polyquaternium-63; Polyquaternium-74; Polyquaternium-76; Polyquatemium-86; Polyquaternium-89; Polyquaternium-95; Polyquaternium-98, Polyquaternium-104; Polyquaternium-111; Polyquaternium-112, and mixtures thereof.

In accordance with yet other embodiments wherein the polycarbodiimide is combined with a cationic polymer, the at least one cationic polymer compound is present in the compositions according to the disclosure in amounts range from about 0.01% to about 20%, and in some embodiments from about 0.01% to about 15%, and in some further embodiments from about 0.05% to about 10%. In some representative embodiments, the cationic polymer is present from about 0.5% to about 5%. In yet other embodiments, the cationic polymer is present from at least 0.5%. And in yet other embodiments, the cationic polymer is present from at least 0.5% to up to about 20%. In some specific embodiments, the cationic polymer is present at about 2%, or about 3%, or about 4%, or about 5%, or about 6%. In some specific embodiments, the cationic polymer is present at about 3% to about 4% In some other specific embodiments, the cationic polymer is present at about 6% to about 8%.

Thus, the cationic polymer may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and therebetween.

Solvent

The composition, according to the present disclosure, further includes suitable solvents for treatment of keratinous fibers. Examples of suitable solvents include water, in some embodiments, distilled or de-ionised, or organic solvents as carriers and solvents for the polycarbodiimides and at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer.

Suitable organic solvents may be chosen from volatile and nonvolatile organic solvents.

Suitable organic solvents are typically C1-C4 lower alcohols, glycols, polyols, polyol ethers, hydrocarbons, and oils. Examples of organic solvents include, but are not limited to, ethanol, isopropyl alcohol, benzyl alcohol, phenyl ethyl alcohol, isododecane, propylene glycol, pentylene glycol, hexylene glycol, glycerol, and mixtures thereof.

Other suitable organic solvents include glycol ethers, for example, ethylene glycol and its ethers such as ethylene glycol monomethyl ether, ethylene glycol monopropyl ether, ethylene glycol monobutyl ether, propylene glycol and its ethers, such as propylene glycol monomethyl ether, propylene glycol monopropyl ether, propylene glycol monobutyl ether, dipropylene glycol and diethylene glycol alkyl ethers, such as diethylene glycol monoethyl ether, diethylene glycolmonobutyl ether, and dipropylene glycol n-butyl ether. Glycol ethers are commercially available from The Dow Chemical Company under the DOW E-series and DOW P-series. An exemplary glycol ether for use in the present invention is dipropylene glycol n-butyl ether, known under the tradename of DOWANOL DPnB.

Suitable organic solvents also include synthetic oils and hydrocarbon oils include mineral oil, petrolatum, and C10-C40 hydrocarbons which may be aliphatic (with a straight, branched or cyclic chain), aromatic, arylaliphatic such as paraffins, iso-paraffins, isododecanes, aromatic hydrocarbons, polybutene, hydrogenated polyisobutene, hydrogenated polydecene, polydecene, squalene, petrolatum and isoparaffins, silicone oils, fluoro oils and mixtures, thereof.

The term “hydrocarbon based oil” or “hydrocarbon oil” refers to oil mainly containing hydrogen and carbon atoms and possibly oxygen, nitrogen, sulfur and/or phosphorus atoms. Representative examples of hydrocarbon based oils include oils containing from 8 to 16 carbon atoms, and especially branched C8 C16 alkanes (also known as isoparaffins), for instance isododecane (also known as 2,2,4,4,6 pentamethylheptane), isodecane and isohexadecane.

Examples of silicone oils that may be useful in the present invention include nonvolatile silicone oils such as polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2 phenylethyl trimethylsiloxysilicates, and dimethicones or phenyltrimethicones with a viscosity of less than or equal to 100 cSt.

Other representative examples of silicone oils that may be useful in the present invention include volatile silicone oils such as linear or cyclic silicone oils, and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of fluoro oils that may be suitable for use in the present invention include volatile fluoro oils such as nonafluoromethoxybutane and perfluoro methylcyclopentane.

Particularly suitable solvents in a composition of the present disclosure include water, isododecane, ethanol, and combinations thereof. The solvent will typically be present in total amounts ranging from about 60% to 98%, in some embodiments, from 80% to 96%, by weight, including all ranges and subranges therebetween, based on the total weight of the composition. Thus, solvents may be present in amounts of about 98%, 97%, 96%, 95%, 94%, 93%, 92%, 91%, 90%, 85%, 80%, 75%, 70%, 65%, 60%, and about 55%, 50%, 48%, 46%, 45%, 44%, 42%, 40%, by weight, based on the total weight of the composition, including increments and ranges therein and there between from about 40% to about 98%.

In yet some other embodiments, the solvent of the present dislcosure does not comprise water and/or organic solvent that is added as a separate ingredient, by itself, into the compositions of the present invention, such that water and/or organic solvent is present in the compositions of the present invention when it accompanies one or more ingredients of a raw material that is added into the compositions of the invention.

When the compositions of the disclosure contain water, according to various embodiments, water can be present in amounts of about 98% or less, such as about 96%, 95%, 90%, 85%, 80%, 75%, 70%, 65%, 60%, 55%, 50%, 48%, 46%, 45%, 44%, 42%, 40%, 35%, 30%, 20%, 10%, or 5% or less, by weight, based on the total weight of the composition, including increments and ranges therein and there between.

When the compositions of the disclosure contain an organic solvent(s), according to various embodiments, the organic solvent(s) can be present in a total amount of about 98% or less, such as about 96%, 95%, 90%, 85%, 80%, 75%, 70%, 65%, 60%, 55%, 50%, 48%, 46%, 45%, 44%, 42%, 40%, 35%, 30%, 20%, 10%, or 5% or less, by weight, based on the total weight of the composition, including increments and ranges therein and there between.

Additives

The composition, according to the present disclosure, further includes suitable additives for treatment of keratinous fibers.

The composition according to the disclosure may also comprise additives chosen from amine or amino compounds (e.g., amino silicones, polyamines, diamines, alkyl monoamines, alkoxylated monoamines, alkoxylated polyamines, and amino functionalized silane compounds), surfactants (anionic, nonionic, cationic and amphoteric/zwtterionic), and polymers other than the polycarbodiimide compounds of the invention such as anionic polymers, nonionic polymers, amphoteric polymers, polymeric rheology modifiers, thickening and/or viscosity modifying agents, associative or non-associative polymeric thickeners. Other suitable additives may be chosen from non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof.

The rheology modifiers and thickening/viscosity-modifying agents that may be employed in compositions of the present disclosure may include any water-soluble or water-dispersible compound that is compatible with the polycarbodiimide, cationic polymer compound s, and compositions of the disclosure, such as acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethyl cellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.

The compositions of certain embodiments may comprise stabilizers, for example sodium chloride, magnesium dichloride or magnesium sulfate.

Amino Compounds

The composition according to the present disclosure comprises one or more amino compounds. The term “amino compound” is intended to mean any compound comprising at least one primary, secondary or tertiary amine or a quaternary ammonium group.

The amino compound of the present disclosure is chosen from alkyl monoamines, alkoxylated polyamines, alkoxylated monoamines, polyamines, and mixtures thereof. In certain embodiments, the amino compounds of the present disclosure do not contain silicon atoms or silicone moieties. In other embodiments, the amino compounds of the present disclosure contain silicon atoms or silicone moieties.

Alkyl Monoamines

The alkyl monoamines of the present disclosure are amino compounds having one amino group.

Non-limiting examples of representative alkyl monoamines include aliphatic amine compounds corresponding to formula (IA) and their salts:

RN(R′)₂ (IA)

wherein:

R is a hydrocarbon radical containing at least 6 carbon atoms. In addition, R can be linear or branched, acyclic or cyclic, saturated or unsaturated, aliphatic or aromatic, substituted or unsubstituted. Typically, R is a linear or branched, acyclic alkyl or alkenyl group or an alkyl phenyl group; and

the groups R′, which may be identical or different, represent H or a hydrocarbon radical containing less than 6 carbon atoms. In addition, the groups R′, which may be identical or different, are linear or branched, acyclic or cyclic, saturated or unsaturated, substituted or unsubstituted. In certain embodiments, the groups R′, which may be identical or different, are H or a methyl group.

Representative alkyl monoamines include, but are not limited to the following examples: dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

Other non-limiting examples of representative alkyl monoamines include amidoamine compounds corresponding to formula (IIA) and their salts:

RCONHR′N(R″)₂ (IIA)

wherein:

R is a hydrocarbon radical containing at least 6 carbon atoms. In addition, R can be linear or branched, acyclic or cyclic, saturated or unsaturated, aliphatic or aromatic, substituted or unsubstituted. Typically, R is a linear or branched, acyclic alkyl or alkenyl group or an alkyl phenyl group; and

R′ is a divalent hydrocarbon radical containing less than 6 carbon atoms, in certain embodiments 2 or 3 carbon atoms, and

R″ is H or a hydrocarbon radical containing less than 6 carbon atoms. In addition, R″ is linear or branched, acyclic or cyclic, saturated or unsaturated, substituted or unsubstituted. Typically, R″ is a linear or branched, acyclic alkyl or alkenyl group. In certain embodiments, R″ is H or a methyl group.

Representative amidoamines include, but are not limited to the following examples: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palmitamidopropyl dimethylamine, stearamidoethyldiethylamine, and mixtures thereof.

Alkoxylated Polyamines

The alkoxylated polyamines of the present disclosure are chosen from amine compounds having at least two amino groups and at least one degree of alkoxylation. The alkoxylation is provided by an alkylene oxide group which is in certain embodiments chosen from ethylene oxide and propylene oxide.

Non-limiting representative examples of suitable alkoxylated polyamines include compounds corresponding to formula (IB):

NH₂R(R′CHCH₂O)ₓ(R′CHCH₂O)_{y}(R′CHCH₂O)_{z}RNH₂ (IB)

wherein R represents a —CH2-, —CH₂CH₂—, —CHCH₃— or —C(CH₃)₂-group, or a hydrocarbon radical containing at least 3 carbon atoms that is linear or branched, acyclic or cyclic, saturated or unsaturated, aliphatic or aromatic, substituted or unsubstituted;

x, y, and z independently of one another, represent numbers of from 0 to about 100;

R′ represents hydrogen, or an alkyl group, in certain embodiments a methyl group; and

the sum of x+y+z is at least 1.

In formula (IB), R is in certain embodiments a linear or branched, acyclic alkyl or alkenyl group or an alkyl phenyl group; x, y, and z independently of one another, in certain embodiments represent numbers ranging from 2 to 100.

Examples of the alkoxylated polyamines for use in the present disclosure which correspond to formula (IB) include, for example, tetradecyloxypropyl-1,3-diaminopropane; a C12-14 alkyl oxypropyl-1,3-diaminopropane; a C12-15 alkyloxypropyl amine and other similar materials that are commercially available from Tomah under the tradename of TOMAH DA-17.

Other examples of alkoxylated polyamines of Formula (IB) are diamine compounds belonging to the JEFFAMINE series such as the JEFFAMINE D and JEFFAMINE ED series available from Huntsman Corporation, Salt Lake City, Utah. Examples of these Jeffamine compounds are JEFFAMINE D230, JEFFAMINE D400, JEFFAMINE D2000, JEFFAMINE D4000, JEFFAMINE HK-511, JEFFAMINE ED600, JEFFAMINE ED900, and JEFFAMINE ED2003. JEFFAMINE D series compounds are amine terminated PPGs (polypropylene glycols) and JEFFAMINE ED series compounds are polyether diamine based with a predominantly PEG (polyethylene glycol) backbone.

Other non-limiting representative examples of suitable alkoxylated polyamines in the diamine form include compounds corresponding to formula (IIB):

NH₂(CH₂)ₓOCH₂CH₂O(CH₂)ₓNH₂ (IIB)

wherein x is 2 or 3.

Examples of alkoxylated polyamines of Formula (IIB) are diamine compounds belonging to the JEFFAMINE series available from Huntsman Corporation, Salt Lake City, Utah, such as JEFFAMINE EDR148, and JEFFAMINE EDR176.

Additional non-limiting representative examples of alkoxylated polyamines in the triamine form include compounds corresponding to formula (IIIB):

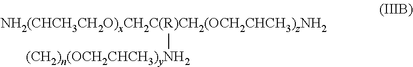

wherein R is hydrogen, —CH₂ or —C2H₅,

n=0 or 1, and

x, y, and z independently of one another, represent numbers of from 0 to 100 and the sum of x+y+z is at least 1.

Examples of alkoxylated polyamines for use in the present disclosure which correspond to formula (IIiB) are triamine compounds belonging to the JEFFAMINE series such as the JEFFAMINE T series available from Huntsman Corporation, Salt Lake City, Utah.

Examples of the JEFFAMINE T series compounds are JEFFAMINE T403, JEFFAMINE T3000, and JEFFAMINE T5000. JEFFAMINE T series compounds are triamines made by reacting PO with a triol initiator followed by aminating the terminal hydroxyl groups.

Another type of representative alkoxylated polyamines include compounds of formulas (IVB) and (VB) hereunder:

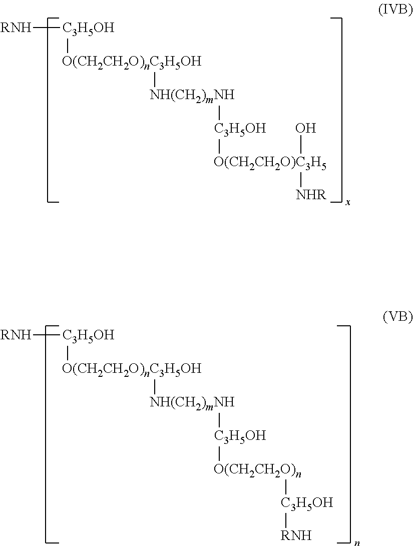

wherein:

R in formula (IVB) represents the alkyl group derived from tallow and R in formula (VB) represents the alkyl group derived from coconut oil;

n in both formulas (IVB) and (VB) has a total value ranging from 10 to 20;

m in both formulas (IVB) and (VB) has a value ranging from 2 to 6; and

x in both formulas (IVB) and (VB) has a value ranging from 2 to 4.

One particular triamine alkoylated polyamine compound is JEFFAMINE T-500 polyetheramine of the formula:

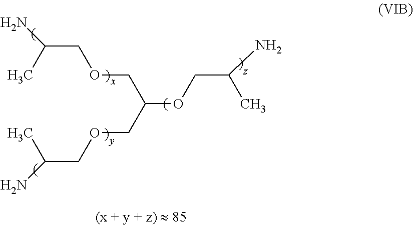 [SMILES: CC(N)COCC(COCC(C)N)OCC(C)N]

Other representative types of alkoxylated polyamines include aminosilicones with at least one degree of alkoxylation.

Representative examples of alkoxylated polyamines for use in the present disclosure include compounds of Formulas (IVB), (VB) and (VIB) above, such as PEG-15 Tallow Polyamine, PEG-15 Cocopolyamine, and JEFFAMINE T-500 polyetheramine, respectively.

Alkoxylated Monoamines

The alkoxylated monoamines of the present disclosure are chosen from amine compounds having at one amino groups and at least one degree of alkoxylation. The alkoxylation is provided by an alkylene oxide group which is in certain embodiments chosen from ethylene oxide and propylene oxide.

Non-limiting representative examples of suitable alkoxylated monoamines include compounds corresponding to the formula (IC):

RN[(R′CHCH₂O)ₓH][(R′CHCH₂O)_{y}H] (IC)

wherein R is a hydrocarbon radical containing at least 6 carbon atoms. R can be linear or branched, acyclic or cyclic, saturated or unsaturated, aliphatic or aromatic, substituted or unsubstituted;

x and y, independently of one another, represent numbers of from 0 to 100 provided that the sum of x+y is >0;

the groups R′, which may be identical or different, represent hydrogen, or an alkyl group such as a methyl group.

Typically, R is a linear or branched, acyclic alkyl or alkenyl group or an alkyl phenyl group; x and y, independently of one another, are each typically a number from 0 to 30.

Typically, one R′ group is hydrogen, and the other one is methyl.

Examples of representative alkoxylated monoamines for use in the present disclosure which correspond to formula (IC) are PEG-2 Cocamine, PEG-3 Cocamine, PEG-5 Cocamine, PEG-10 Cocamine, PEG-15 Cocamine, PEG-20 Cocamine, PEG-2 Lauramine, PEG-12 Palmitamine, PEG-2 Rapeseedamine, PEG-2 Oleamine, PEG-5 Oleamine, PEG-6 Oleamine, PEG-10 Oleamine, PEG-15 Oleamine, PEG-20 Oleamine, PEG-25 Oleamine, and PEG-30 Oleamine. Other examples are alkoxylated derivatives of soyamine, stearamine and tallow amine.

Other non-limiting examples of suitable alkoxylated monoamines include compounds corresponding to formula (IIC):

RNR″[(R′CHCH₂O)ₓH] (IIC)

wherein R is a hydrocarbon radical containing at least 6 carbon atoms. R can be linear or branched, acyclic or cyclic, saturated or unsaturated, aliphatic or aromatic, substituted or unsubstituted;

x represents a number of from 1 to 100;

R′ represents hydrogen, or an alkyl group such as in particular a methyl group; and

R″ is a hydrogen or a hydrocarbon radical.

Typically, R is a linear or branched, acyclic alkyl or alkenyl group or an alkyl phenyl group; x is typically a number from 1 to 30.

When R″ in formula (IIC) is a hydrocarbon radical group, this group may be linear or branched, saturated or unsaturated, substituted or unsubstituted. The hydrocarbon radical represented by R″ may also contain an alkoxylated moiety (as defined by [(R′CHCH2O)yH]), and/or heteroatoms such as nitrogen. When R″ contains at least one alkoxylated moiety, the total number of alkoxylation in the formula may range from 1 to 120.

Examples of alkoxylated monoamines for use in the present disclosure which correspond to formula (IIC) are PEG-3 Tallow Aminopropylamine, PEG-10 Tallow Aminopropylamine, PEG-15 Tallow Aminopropylamine, and PEG-105 Behenyl Propylenediamine.

Additional non-limiting examples of alkoxylated monoamines include compounds corresponding to formula (IIIC):

R(R′CHCH₂O)ₓ(R′CHCH₂O)_{y}NH₂ (IIIC)

wherein R is a hydrocarbon radical containing at least 6 carbon atoms. R can be linear or branched, acyclic or cyclic, saturated or unsaturated, aliphatic or aromatic, substituted or unsubstituted;

x and y, independently of one another, represent numbers of from 0 to 100 with the proviso that the sum of x+y is >0;

the groups R′, which may be identical or different, represent hydrogen, or an alkyl group such as in particular a methyl group.

Typically, R is a linear or branched, acyclic alkyl or alkenyl group or an alkyl phenyl group; x and y, independently of one another, are each typically a number from 0 to 30.

Examples of alkoxylated monoamines for use in the present disclosure which correspond to formula (IIIC) are polyetheramines containing a monoamine group. These polyetheramines are commercially available from Hunstman under the tradename JEFFAMINE (M series such as M-600, M-1000, M-2005 and M-2070) and SURFONAMINE series (B-60, B-100, B-200, L-100, L-200, L-207, L-300).

Polyamines

The polyamines may in particular be chosen from polyvinylamines, aminated polysaccharides, amine substituted polyalkylene glycols, amine substituted polyacrylate crosspolymers, amine substituted polyacrylates, amine substituted polymethacrylates, proteins, protein derivatives, amine substituted polyesters, polyamino acids, polyalkylamines, diethylene triamine, triethylenetetramine, spermidine, spermine and mixtures thereof. The polyamines for use in the present disclosure can also be chosen from aminosilicones having at least two amino groups.

The amino compound of the present disclosure selected from polyamines may be chosen from amine-containing polymers, in particular having a weight-average molecular weight ranging from 500 to 1,000,000, in certain embodiments ranging from 500 to 500,000, and preferentially ranging from 500 to 100,000. As amine-comprising polymer, use may be made of polyamines such as poly((C2-C5)alkyleneimines), and in particular polyethyleneimines and polypropyleneimines, especially poly(ethyleneimine)s; poly(allylamine); polyvinylamines and copolymers thereof, in particular with vinylamides; polyamino acids which have NH₂ groups; aminodextran; amino polyvinyl alcohol; acrylamidopropylamine-based copolymers; and chitosans.

The polyamines of the present disclosure are also in certain embodiments chosen from polyvinylamines which are generally sold under the trade name LUPAMINE or LUVIQUAT from BASF. One representative example of such polyamines are polyvinylamines sold under the LUVIQUAT series such as Vinylamine/vinylformamide copolymer (INCI name), sold as LUVIQUAT 9030 by BASF.

The polyamines of the present disclosure may also be chosen from Vinylamine/Vinyl Alcohol Copolymer (INCI name).

Other representative polyamines of the present disclosure include are amine substituted polyalkylene glycols such as PEG-15 cocopolyamine and PEG-15 Tallow Polyamine and amine substituted polyacrylate crosspolymer such as the product sold under the name CARBOPOL AQUA CC polymer by Lubrizol Advanced Materials, Inc.

The polyamine compound of the present disclosure may also be chosen from proteins and protein derivatives such as wheat protein, soy protein, oat protein, collagen, and keratin protein.

In an embodiment of the present disclosure, the polyamine compound is chosen from polyamino acid compounds comprising lysine, compounds comprising arginine, compounds comprising histidine, and compounds comprising hydroxylysine. Non limiting examples include chitosan and polyamino acids such as polyarginine, polyhistidine, polylysine, and mixtures thereof.

In one representative embodiment of the present disclosure, amino compound of the present disclosure is chosen from polyvinylamines such as Vinylamine/vinylformamide copolymer (INCI name), sold as LUVIQUAT 9030 by BASF; alkoxylated polyamines which correspond to formula (IIIB) such as JEFFAMINE T403, JEFFAMINE T3000, and JEFFAMINE T5000 (in particular, corresponds to formula (VIB); alkoxylated monoamines which correspond to formula (IIIC) such as JEFFAMINE M-600; and alkyl amines selected from aliphatic amines and amidoamines; and mixtures, thereof.

The alkyl amines selected from aliphatic amines are in certain embodiments chosen from dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine, and mixtures thereof.

The alkyl amines selected from amidoamines are in certain embodiments chosen from stearamidopropyl dimethylamine, isostearamidopropyl dimethylamine, stearamidoethyl dimethylamine, lauramidopropyl dimethylamine, behenamidopropyl dimethylamine, dilinoleamidopropyl dimethylamine, babassuamidopropyl dimethylamine, cocamidopropyl dimethylamine, stearamidoethyldiethylamine, and mixtures thereof.

Silicone Polymers Having At Least One Carboxylic Acid Group (Carboxysilicone Polymers).

The silicone polymers having at least one carboxylic acid group, referred herein as carboxysilicone polymers, according to the present disclosure, may be an organopolysiloxane comprising:

(A) a compound having the following formula:

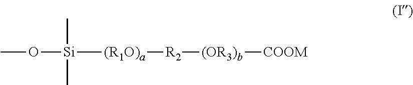

wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene, or

(B) a group comprising at least one pyrrolidone carboxylic acid unit having the following formula:

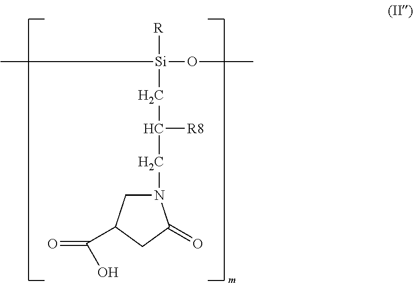

in which R is selected from methyl or phenyl; R8 is hydrogen or methyl, m is an integer from 1 to 1000, or

(C) a group comprising at least one polyvinyl acid/ester unit (C) resulting from the polymerization of Divinyl-PDMS, Crotonic Acid, Vinylacetate, and Vinyl Isoalkylester,

and combinations of (A), (B) and (C).

Suitable carboxysilicone polymers include, for example, a silicone polymer comprising at least one carboxylic acid group chosen from organopolysiloxanes of formula:

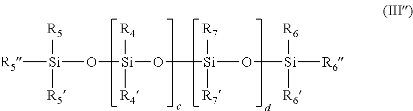

wherein the radicals R4, R4′ are identical to or different from each other and are chosen from a linear or branched C1-C22 alkyl radical, a C1-C22 alkoxy radical and a phenyl radical, the radicals R5, R5′, R5″, R6, R6′, R6″, R7, and R7′ are identical to or different from each other and are chosen from a linear or branched C1-C22 alkyl radical, a C1-C22 alkoxy radical, a phenyl radical, a radical —(R1O)a —R2-(OR3)b —COOM, a radical containing pyrrolidone carboxylic acid, a radical of polyvinyl acid/ester; and

wherein at least one of the radicals R5, R6 and R7 is a radical chosen from a radical —(R1O)a —R2-(OR3)b —COOM, a radical containing pyrrolidone carboxylic acid, a radical of polyvinyl acid/ester;

wherein R1, R2, R3, a, b and M have the same meaning as described in Unit (A) above;

wherein c and d are integers from 0 to 1000, the sum c+d in some particular embodiments ranging from 1 to 1000 or from 2 to 1000.

Among the carboxysilicone polymers of formula (III″) that comprise at least one unit (I″), which in some particular embodiments are the compounds of formula below:

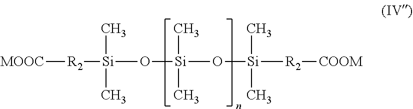

wherein R2, and M have the same meaning as described in Unit (A) above, n is an integer from 1 to 1000. Examples are: dual-end carboxy silicones X-22-162C from Shin Etsu and Silform INX (INCI name: Bis-Carboxydecyl Dimethicone) from Momentive.

Other exemplary embodiments organopolysiloxanes of formula (III″) are the ones of formula:

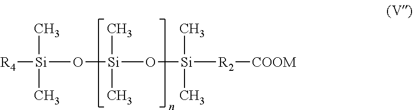

in which R₂, R₄, n, and M having the same meaning as in Unit above. An example is a single-end carboxy silicone X-22-3710 from Shin Etsu.

Other exemplary embodiments organopolysiloxanes of formula (III″) are the ones of formula:

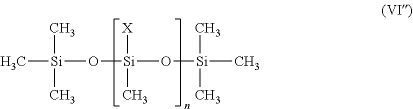

wherein X is a radical —(R1O)a —R2-(OR3)b —COOM wherein R1, R2, R3, a, b and M have the same meaning as described in Unit (A) above.

Even more particularly, the compounds of formula (VI′) in which a and b are equal to 0 and R2 is a linear or branched C2-C12 alkylene group such as (CH2)9, (CH2)10 or —CH(CH3)- are exemplary embodiments. An example is a side-chain carboxy silicone X-22-3701E from Shin Etsu.

Among the organopolysiloxanes of formula (III″) that contain unit (B), exemplary embodiments include the compounds of formula below:

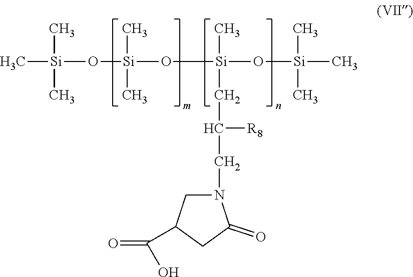

wherein R8, m, are defined as in Unit (B) above and n is an interger from 1 to 1000. An example is Grandsil PCA such as in Grandsil SiW-PCA-10 (INCI name: Dimethicone (and) PCA Dimethicone (and) Butylene Glycol (and) Decyl Glucoside from Grant Industries.

Among the organopolysiloxanes of formula (III″) that contain polyvinyl acid/ester Unit (C), exemplary embodiments are crosslinked anionic copolymers comprised of organic polymer blocks and silicone blocks, resulting in a multiblock polymer structure. In particular, the silicone-organic polymer compound of the present invention may be chosen from crosslinked anionic copolymers comprising at least one crosslinked polysiloxane structural unit. An example of such a branched multi-block carboxysilicone polymer is Belsil® P1101 (may also be known under the tradename Belsil® P1101) (INCI name: Crotonic Acid/Vinyl C8-12 Isoalkyl Esters/VA/Bis-Vinyldimethicone Crosspolymer, also known by the technical name of Crotonic Acid/Vinyl C8-12 Isoalkyl Esters/VA/divinyldimethicone Crosspolymer) from Wacker Chemie AG.

Additional suitable carboxysilicone polymers are described, for example, in patent applications WO 95/23579 and EP-A-0,219,830, which are hereby incorporated by reference in their entirety.

Compounds corresponding to formula (VI″) above are sold, for example, under the name HUILE M 642 by the company Wacker, under the names SLM 23 000/1 and SLM 23 000/2 by the company Wacker, under the name 176-12057 by the company General Electric, under the name FZ 3703 by the company OSI and under the name BY 16 880 by the company Toray Silicone.

Other non-limiting examples of carboxysilicone polymers are silicone carboxylate containing polymers (silicone carboxylates).

Suitable silicone carboxylates may be chosen from water soluble silicone compounds comprising at least one carboxylic acid group, oil soluble silicone compounds comprising at least one carboxylic acid group, water-dispersible silicone compounds comprising at least one carboxylic acid group, and silicone compounds comprising at least one carboxylic acid group which are soluble in organic solvents. In one embodiment, the silicone carboxylate further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups and propylene oxide groups.

The at least one carboxylic acid group may be chosen from terminal carboxylic acid groups and pendant carboxylic acid groups. Further, the at least one carboxylic acid may be chosen from carboxylic acid groups in free acid form, i.e., —COOH, and carboxylic acid groups in salt form, i.e., —COOM, wherein M may be chosen from inorganic cations, such as, for example, potassium cations and sodium cations, and organic cations.

In one embodiment, the silicone carboxylate is a compound of formula:

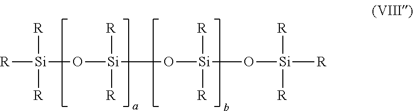

wherein a is an integer ranging from 1 to 100; b is an integer ranging from 0 to 500; and R, which may be identical or different, are each chosen from optionally substituted hydrocarbon groups comprising from 1 to 9 carbon atoms, optionally substituted phenyl groups, and groups of the following formula:

—(CH₂)₃—O-(EO)_{c}—(PO)_{d}-(EO)ₑ—C(O)—R′—C(O)—OH (IX″)

wherein c, d, and e, which may be identical or different, are each integers ranging from 0 to 20; EO is an ethylene oxide group; PO is a propylene oxide group; and R′ is chosen from optionally substituted divalent hydrocarbons, such as alkylene groups and alkenylene groups comprising from 2 to 22 carbon atoms, and optionally substituted divalent aromatic groups, such as groups of the following formula:

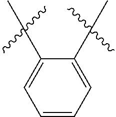 [SMILES: *c1ccccc1*]

and groups of the following formula:

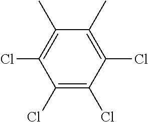

with the proviso that at least one of the R groups is chosen from groups of formula (XIV) and with the further proviso that when only one of the R groups is chosen from groups of formula (XIV), the other R groups are not all methyl groups.

Non-limiting examples of silicone carboxylates include those commercially available from Noveon under the name Ultrasil® CA-1 Silicone (Dimethicone PEG-7 Phthalate) and Ultrasil® CA-2 Silicone (Dimethicone PEG-7 Succinate), both of which correspond to formula (XV) below. Thus, in one embodiment, the at least one silicone carboxylate is chosen from a compound of formula below and salts thereof:

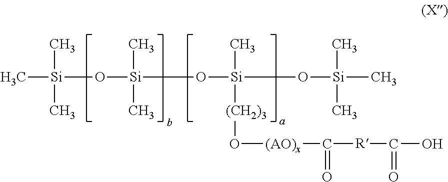

wherein a is an integer ranging from 1 to 100, b is an integer ranging from 0 to 500, AO is chosen from groups of the following formula:

-(EO)c—(PO)d-(EO)e-

wherein c, d, and e, which may be identical or different, are each integers ranging from 0 to 20; EO is an ethylene oxide group; PO is a propylene oxide group; x is an integer ranging from 0 to 60; R′ is chosen from optionally substituted divalent hydrocarbons, such as alkylene groups and alkenylene groups comprising from 2 to 22 carbon atoms, and optionally substituted divalent aromatic groups, such as groups of the following formula:

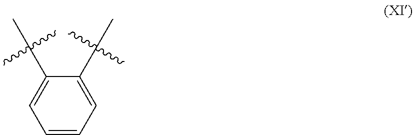 [SMILES: *c1ccccc1*]

and groups of formula

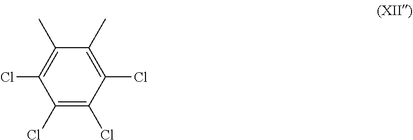

Non-limiting examples of silicone carboxylates include those described in U.S. Pat. Nos. 5,248,783 and 5,739,371, the disclosures of which are incorporated herein by reference, and which are silicone compounds of formula (XII″).

Latex Polymers

According to various exemplary embodiments, the compositions of the present invention can further comprise one or more latex polymers (also referred to as “latex polymers” in this application) can be chosen from carboxyl functional acrylate latex polymers, carboxyl functional polyurethane latex polymers, carboxyl functional silicone latex polymers, carboxyl functional non-acrylate latex polymers and mixtures thereof.

In various embodiments, the latex polymers of the present invention can be film-forming latex polymers or non film-forming latex polymers.

In at least certain embodiments of the disclosure, the latex polymers are provided in the form of aqueous dispersions prior to formulating the compositions of the disclosure. In various embodiments, the aqueous dispersions may be obtained through an emulsion polymerization of monomers wherein the resulting latex polymers have a particle size lower than about 1 micron. In at least one exemplary embodiment, a dispersion prepared by the polymerization in water of one or more monomers having a polymerizable double bond may be chosen. In another exemplary embodiment, the aqueous dispersions obtained through an emulsion polymerization may be spray-dried.

In other embodiments, the latex polymers are produced from condensation reactions between monomers and subsequently dispersed in an aqueous medium.

Thus, the latex polymers may, in various exemplary embodiments, exist as dispersed polymer particles in a dispersion medium, such as an aqueous dispersion medium. The latex polymers may, in various embodiments, each be dispersed in independent dispersion media or dispersed together in the same dispersion medium.

The dispersion medium comprises at least one solvent chosen from water. The dispersion medium may further comprise at least one solvent chosen from cosmetically acceptable organic solvents such as those described above.

In embodiments according to the disclosure, the latex polymer particles are not soluble in the solvent of the dispersion medium, i.e. are not water soluble and/or are not soluble in the at least one cosmetically acceptable organic solvent. Accordingly, the latex polymers retain their particulate form in the solvent or solvents chosen.

In at least certain exemplary embodiments, latex polymer particles according to the disclosure may have an average diameter ranging up to about 1000 nm, such as from about 50 nm to about 800 nm, or from about 100 nm to about 500 nm. Such particle sizes may be measured with a laser granulometer (e.g. Brookhaven BI90).

In various embodiments, the latex polymers may, independently, be neutralized, partially neutralized, or unneutralized. In exemplary embodiments where the latex polymers are neutralized or partially neutralized, the particle size may be, for example, greater than about 800 nm. In at least certain embodiments, the particulate form of the latex polymers is retained in the dispersion medium.

In further embodiments, the latex polymers may be chosen from uncharged and charged latex polymers. Thus, the latex polymers may, according to various exemplary embodiments, be chosen from nonionic latex polymers, cationic latex polymers, anionic latex polymers and amphoteric latex polymers.

By way of non-limiting example only, the latex polymers may be chosen from carboxyl functional acrylate latex polymers, such as those resulting from the homopolymerization or copolymerization of ethylenically unsaturated monomers chosen from vinyl monomers, (meth)acrylic monomers, (meth)acrylamide monomers, mono- and dicarboxylic unsaturated acids, esters of (meth)acrylic monomers, and amides of (meth)acrylic monomers. The term “(meth)acryl” and variations thereof, as used herein, means acryl or methacryl.

The (meth)acrylic monomers may be chosen from, for example, acrylic acid, methacrylic acid, citraconic acid, itaconic acid, maleic acid, fumaric acid, crotonic acid, and maleic anhydride. The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, ca-methyl styrene, chlorostyrene, alkylstyrene, divinylbenzene and diallyl phthalate, as well as para-styrensulfonic, vinylsulfonic, 2-(meth)acryloyloxyethylsulfonic, 2-(meth)acrylamido-2-methylpropylsulfonic acids, and mixtures thereof.

The list of monomers given is not limiting, and it should be understood that it is possible to use any monomer known to those skilled in the art which includes acrylic and/or vinyl monomers (including monomers modified with a silicone chain).

In at least certain, non-limiting exemplary embodiments carboxyl functional acrylate latex polymers may be chosen from aqueous dispersions of Methacrylic Acid/Ethyl Acrylate copolymer (INCI: Acrylates Copolymer, such as Luviflex® SOFT by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as Fixate Superhold™ by Lubrizol), Styrene/Acrylic copolymer (such as Acudyne Shine by Dow Chemical), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as Daitosol 5000AD, Daito Kasei Kogyo), Acrylates Copolymers, such as those known under the tradenameDermacryl AQF (Akzo Nobel), under the tradename LUVIMER® MAE (BASF), or under the tradename BALANCE CR (AKZO NOBEL), Acrylates/Hydroxyesters Acrylates Copolymer, known under the tradename ACUDYNE 180 POLYMER (Dow Chemical), Styrene/Acrylates Copolymer, known under the tradename Acudyne Bold from Dow Chemical, Styrene/Acrylates/Ammonium Methacrylate Copolymer, known under the tradename SYNTRAN PC5620 CG from Interpolymer, and mixtures thereof.

In yet further exemplary and non-limiting embodiments, the latex polymers may be chosen from carboxyl functional polyurethane latex polymers, such as aqueous polyurethane dispersions. These polyurethanes are conventionally formed by the reaction of prepolymer (i) with a coreactant (ii) to produce a carboxyl terminated or pendant polyurethane polymer. The prepolymer (i) may have the structure according to the formula (I″):

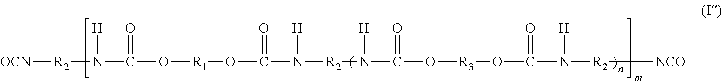

wherein R1 is chosen from bivalent radicals of a dihydroxyl functional compound, R2 is chosen from hydrocarbon radicals of an aliphatic or cycloaliphatic polyisocyanate, and R3 is chosen from radicals of a low molecular weight diol, optionally substituted with ionic groups or potential ionic groups, n ranges from about 0 to about 5, and m is greater than about 1.

Suitable dihydroxyl compounds for providing the bivalent radical R1 include those having at least two hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the disclosure.

Optional polyisocyanates for providing the hydrocarbon-based radical R2 include, for example, organic diisocyanates having a molecular weight ranging from about 100 to about 1500, such as about 112 to about 1000, or about 140 to about 400.

Optional diisocyanates are those chosen from the general formula R2(NCO)2, in which R2 represents a divalent aliphatic hydrocarbon group comprising from about 4 to 18 carbon atoms, a divalent cycloaliphatic hydrocarbon group comprising from about 5 to 15 carbon atoms, a divalent aromatic hydrocarbon group comprising from about 7 to 15 carbon atoms, or a divalent aromatic hydrocarbon group comprising from about 6 to 15 carbon atoms.

The use of diols, for example low molecular weight diols, R3, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In certain exemplary embodiments, the compounds contain only aliphatic groups. The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxycyclohexyl)propane), and mixtures thereof. For example, R3 may be derived from neopentyl glycol.

Optionally, the low molecular weight diols may contain ionic or potentially ionic groups. Suitable low molecular weight diols containing ionic or potentially ionic groups may be chosen from those disclosed in U.S. Pat. No. 3,412,054. In various embodiments, compounds may be chosen from dimethylolbutanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In at least certain exemplary and non-limiting embodiments, the low molecular weight diols containing ionic or potentially ionic groups are not used.

Coreactants (ii) are compounds containing functional groups such as hydroxy or amine groups, in certain embodiments primary amine, adapted to react with isocyanate groups in preference to the carboxyl group according to the formula (II″):

X—R4-X X═OH,NH2, (II″)

wherein R₄ represents a divalent aliphatic or cycloaliphatic or aromatic hydrocarbon group, optionally substituted with ionic groups or potentially ionic groups. In various embodiments, compounds may optionally be chosen from alkylene diamines, such as hydrazine, ethylenediamine, propylenediamine, 1,4-butylenediamine and piperazine; In various embodiments, compounds may optionally be chosen from alkylene diols, such as ethylene glycol, 1,4-butanediol (1,4-BDO or BDO), 1,6-hexanediol.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Special compounds may be chosen from dimethylolbutanoic acid (DMBA), dimethylolpropionic acid (DMPA), or carboxyl functional polyester comprising excess equivalents of dicarboxylic acid reacted with lesser equivalents of glycol or carboxyl-containing caprolactone polyester diol.

R1, R2, R3, R4 can have at least one carboxyl group independently.

By way of non-limiting example, such latexes include, but are not limited to, aqueous polyurethane dispersion of Isophthalic Acid/Adipic Acid/Hexylene Glycol/Neopentyl glycol/Dimethylolpropanoic Acid/Isophorone Diisocyanate copolymer (INCI name: Polyurethane-1, such as Luviset® P.U.R, BASF), a copolymer of hexylene glycol, neopentyl glycol, adipic acid, saturated methylene diphenyldiisocyanate and dimethylolpropanoic acid monomers (INCI name: polyurethane 2), a copolymer of PPG-17, PPG-34, isophorone diisocyanate and dimethylolpropanoic acid monomers (INCI name: polyurethane 4), a copolymer of isophthalic acid, adipic acid, hexylene glycol, neopentyl glycol, dimethylolpropanoic acid, isophorone diisocyanate and bis-ethylaminoisobutyl-dimethicone monomers (INCI name: polyurethane 6), Isophorone diisocyanate, cyclohexanedimethanol, dimethylol butanoic acid, polyalkylene glycol and N-methyl diethanolamine copolymer (INCI name: polyurethane 10), Trimethylolpropane, neopentyl glycol, dimethylol propionic acid, polytetramethylene ether glycol and isocyanato methylethylbenzene copolymer (INCI name: polyurethane 12), Isophorone diisocyanate, dimethylol propionic acid, and 4,4′-isopropylidenediphenol reacted with propylene oxide, ethylene oxide and PEG/PPG-17/3 copolymer (INCI name: polyurethane 14), Isophorone diisocyanate, adipic acid, triethylene glycol and dimethylolpropionic acid copolymer (INCI name: polyurethane 15), 2-Methyl-2,4-pentanediol, polymer with 2,2-dimethyl-1,3-propanediol, hexanedioic acid, methylenedicyclohexanediisocyanate and 2,2-di(hydroxymethyl)propanoic acid, hydrolysed, tris(2-hydroxyethyl)amine salts, reaction products with 1,2-ethanediamine (INCI name: polyurethane 17), Polyurethane-27 is a complex polymer that is formed by the reaction of Polyperfluoroethoxymethoxy Difluorohydroxyethyl Ether and isophorone diisocyanate (IPDI) to form a prepolymer. The prepolymer is further reacted with the triethylamine salt of 3-hydroxy-2-(hydroxymethyl)-2-methyl-1-propionic acid (INCI name: polyurethane 27), a complex polymer formed by reacting dimethylolpropionic acid and a polyester composed of Adipic Acid, Hexylene Glycol, Neopentyl Glycol with methylene dicyclohexyldiisocyanate (SMDI) to form a prepolymer. The prepolymer is neutralized with triethylamine and then chain-extended with hydrazine (INCI name: polyurethane 33).

If present in the composition, the above-described additives are generally present in an amount ranging up to about 95% by weight including all ranges and subranges therebetween, based on the total weight of the composition, such as up to about 50%, up to about 40%, up to about 30%, up to about 20%, up to about 15%, up to about 10%, up to about 5%, such as from about 0.001% to about 50%, or from about 0.001% to about 40%, or from about 0.001% to about 30%, or from about 0.001% to about 20%, or from about 0.001% to about 10%, by weight, based on the total weight of the composition.

Needless to say, a person skilled in the art will take care to select this or these optional additional compound(s), and/or the amount thereof, such that the advantageous properties of the composition, according to the invention, are not, or are not substantially, adversely affected by the envisaged addition.

Methods of Preparation and Methods of Use

In some embodiments, the compositions of the present disclosure are prepared by combining the polycarbodiimide, and at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer.

In other embodiments, the compositions of the present disclosure are prepared by combining the polycarbodiimide, at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer, and a solvent chosen from water, organic solvents, and mixtures thereof.

In yet other embodiments, the compositions of the present disclosure are prepared by combining the polycarbodiimide, at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer, and a solvent comprising water and organic solvents.

One-Step Application Process

In one embodiment, a composition including at least one polycarbodiimide and at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer, according the present disclosure, is applied onto keratinous substrates such as hair in a one-step application process. The composition for use in the one-step application is prepared by combining the ingredients, including the polycarbodiimide, and at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer, and solvent, resulting in a composition that is then applied onto the keratinous substrate.

In one embodiment, when the composition for a one-step application process comprises a solvent comprising water and an organic solvent, the polycarbodiimide is combined with water to form an aqueous phase and the at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer is combined with a suitable solvent to form a second active phase, which is in some examples a non-aqueous phase. Both phases are then combined and agitated to form an emulsion. The formed emulsion is then applied to a keratinous substrate such as hair for treatment.

In another embodiment, the polycarbodiimide and at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer are each or together combined with water to form on an aqueous composition which is applied to a keratinous substrate such as hair for treatment.

Two-Step Application Process

In other embodiments, the composition of the present disclosure is applied onto keratinous substrates such as hair in a two-step application process. In a two-step application process, the individual components (polycarbodiimide combined with a solvent and at least one additional active selected from one or more a carboxylic acid compound chosen from fatty acids, their salts, and mixtures thereof, a carboxylic acid compound other than one that is chosen from fatty acids, their salts, and mixtures thereof, and a cationic polymer combined with a solvent) are applied to the keratinous substrate in a step-wise fashion in any order to treat the substrate.

The application of the composition onto a keratinous substrate such as hair, according to the present disclosure, may occur at room temperature.

In other embodiments, the application of the composition according to the present disclosure, may occur at an elevated temperature (or temperatures greater than room temperature) by applying heat to the hair or exposing hair to elevated temperatures. While not so limited, heating may be provided, for example, by commonly used heating tools for example a helmet dryer or blow dryer (40° C. and above) or hot iron or flat iron (120° C.-250° C.) or steam/hot rollers.

The composition and treatment, according to the present disclosure, provides advantageous properties to keratinous fibers. In one embodiment, the composition and treatment, according to the present disclosure, provides hydrophobicity or imparts hydrophobicity to hydrophilic or damaged keratinous fibers, such as damaged hair (platinum bleached), upon application thereto. In certain embodiments, hydrophobicity is provided at room temperature, i.e., without heating or applying heat to the hair. In other embodiments, the hydrophobicity is provided when heat is used on the hair (before or after applying the composition on the hair or during the application of the composition on the hair). In one embodiment, the hydrophobicity provided to less hydrophobic or hydrophilic keratinous fibers includes a contact angle of greater than 50° or greater than 60° or greater than 70°, or ranging from between about 50° and about 80°, such as from between about 60° and about 80°, or such as from between about 70° and about 80°. In some embodiments, the hydrophobicity provided to less hydrophobic or hydrophilic keratinous fibers includes a contact angle of greater than 50° or greater than 60° or greater than 70° or greater than 80° or greater than 90°.

For example, hair treated with the inventive compositions, when heated at 50° C. for 30 minutes and then allowed to cool down for a period of time at room temperature, exhibited increased percent curl retention indicating higher resistance to high humidity and high temperature compared to hair treated with either material alone. With the use of heat (drying in the oven), the percent curl retained using the inventive composition was greater than that obtained when heat was not applied or employed on hair. Accordingly, the composition, according to the present disclosure, provides increased curling benefits when utilized, particularly when the keratinous fibers are exposed to commonly used heating tools, such as a blow dryer (60° C.) or hot iron or flat iron (120° C.-250° C.) or a heat lamp, a heat wand, or other similar devices.

The method or process of using the compositions of the present invention may depend on the type of hair being targeted and, consequently, on the specific ingredients contained in the composition used to style or shape or maintain the shape of hair.

An embodiment of the present invention is a method of styling or shaping or maintaining the shape of hair.

Another embodiment of the present invention is a method of imparting durable or long-lasting style or shape to hair comprising applying onto the hair, any one of the compositions of the present disclosure.

According to at least one embodiment, such a method comprises applying to the hair, an effective amount of any one of the compositions of the present disclosure.

The compositions of the present disclosure may be employed in an effective amount to adequately cover the surface of the fibers of the hair and to achieve a desirable or effective style or shape of the hair as well as a desirable degree of hold. The precise amount of composition to be applied onto the hair will thus depend on the degree of treatment/styling/shaping/hold desired.

An effective amount of the composition is typically from about 0.1 gram to about 50 grams, and in some applications for treatment of hair, in amounts from about 20 to 60 grams, and in yet further embodiments for an abundance of hair in amounts from about 20 to about 80 grams or more. It will thus be appreciated that the amounts applied depend on the amount or volume of keratinous material, such as hair, to be treated and may thus fall within lower ranges for small amounts or patches of hair to the higher ranges and beyond for large amounts or patches of hair. Typical applications are to the whole head in the case of treatment of hair. It will be understood that application to the hair typically includes working the composition through the hair.

Further disclosed herein is the use of the compositions of the present disclosure for shaping or styling hair and/or retaining a hairstyle. Also disclosed is the use of the compositions of the present disclosure for caring for the hair such as for hair repair treatments, or for reducing damage to the hair or for improving the feel of the hair by imparting hydrophobicity to the hair.

The compositions may be applied to wet or dry hair, before or after shaping. They may be used in a non-rinse fashion. In some other embodiments, the composition may be rinsed from the hair.

The hair that has been contacted with the compositions of the present invention may be air-dried and/or further styled or shaped by applying heat on the hair and/or by combing or brushing or running the fingers through the hair. Other shaping tools may be chosen from combs and brushes.

In certain embodiments, the composition is allowed to remain (leave-on time) on the keratin fibers, for example, from about 1 to about 60 minutes, or such as from about 5 to about 45 minutes, or such as from about 5 to about 30 minutes, or such as from about 10 to about 20 minutes, or such as at about 20 minutes, or such as at about 10 minutes.

The smoothing action may be accomplished by use of suitable devices for brushing or smoothing the hair include a hair brush, comb, or flat iron. The smoothing action on the hair may also include running the fingers through the hair.

A suitable applicator device is an applicator brush. It will be appreciated that while a brush is an example of a suitable applicator, particularly for hair, other applicators may be used, including but not limited to spray bottles, squeeze bottles, one and two chamber pumps, tubes, combs, and other applicators known in the art.

Heat (at a temperature of at least 40° C.) can be applied to the hair while the smoothing action is performed on the hair. The heat source can be chosen from a blow dryer, a flat iron, a hair dryer, a heat lamp, a heat wand, or other similar devices.

In addition, independently of the embodiment use, the composition present on the fibers or hair is left in place for a time, generally, from about 1 to about 60 minutes, such as from about 5 to about 45 minutes, or such as from about 5 to about 20 minutes, or such as from about 10 to about 20 minutes, or such as of about 20 minutes or such as of about 10 minutes. In alternate embodiments, the treatment times may be longer, and in some embodiments, appreciably longer, such that the application may be left on for up to 24 hours to about 48 hours.

The compositions of the present invention are easy to spread on hair.

It has surprisingly and unexpectedly discovered that the application of the composition onto the hair results in the retention of the shape or style or curl of hair or of making the hair humidity resistant. It was also surprisingly and unexpectedly discovered that the application of the composition onto the hair results in improving the quality of the hair, for example, better hair feel and appearance.

The shape/styling control, the curl retention, humidity resistant, and hair care effects obtained using the compositions and methods of the present invention may also be durable or long-lasting, i.e., wash or shampoo resistant.

As used herein, “long-lasting” or “durable” is understood to mean that the benefits imparted to hair by the compositions of the invention last over a period of time and/or over high humidity conditions and/or after one or multiple wash cycles (with water or shampoo/water or shampoo/water/conditioner/water or conditioner/water). The multiple wash cycles is understood to mean more than one wash cycle, such as two or three or four or five or six or seven or eight or nine or ten wash cycles.

Another embodiment of the present invention is method for imparting durable or long-lasting style/shape and/or curl and/or care to hair comprising (a) providing the composition of the present invention, and (b) providing instructions for applying the composition to the hair.

Instructions for applying the composition of the present invention onto keratinous substrates such as hair on the head or eyelashes may comprise directions of use of the composition for the end-user to follow. The end-user may be a consumer or cosmetologist or salon hair dresser. Directions may comprise instructing the end-user to take an amount of the composition in sufficient quantity such that the composition adequately covers the hair fibers and imparts the desired shape or style or hold to the hair fibers. Directions may additionally instruct the end-user to use a device such as a comb, brush (e.g., hair brush or brush wand), flat iron plates, blow dryer or the fingers for shaping or styling the hair or for separating the fibers of the hair. Directions may also additionally instruct the end-user to apply heat to the hair such as by blow drying the hair or using a heating device on the hair.

Instructions for applying the composition of the present invention onto keratin fibers such as hair may appear on the container (such as can, bottle or jar) holding the composition of the present invention or on the box or carton or other packaging comprising the container holding the composition.

Another embodiment of the present invention is method protecting a keratinous fiber chosen from hair comprising applying to the keratinous fiber the composition of the present invention in an amount effective to protect or repair said keratinous fiber before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

The compositions described above are useful for application onto keratinous substrates such as hair on the head of human individuals.

Thus, the compositions of the present invention can be made into various cosmetic products such hair care products, hair styling products and make up products.

Representative types of hair care compositions, including hair cosmetic and styling compositions, of the present invention include compositions for shaping the hair, maintaining the shape of the hair, styling products (e.g., gels, creams, milks, pastes, waxes, ointments, serums, foams, hair lotions, mousses, pump-sprays, non-aerosol sprays and aerosol sprays), conditioning or protection from heat damage, leave-in hair treatments, rinse-off hair treatments, combination shampoo/styling compositions and hair volumizing compositions.

The compositions of the present invention can be in the form of an aqueous composition or an emulsion, such as a lotion or cream, and in some embodiments may be applied in another form, such as in a serum such as an anhydrous serum.

In one embodiment, the composition of the present invention is in the form of a non-aerosol spray, in some embodiments, containing a volatile organic solvent/compound.

In one embodiment, the composition of the present invention is in the form of a cream.

In another embodiment, the composition of the present invention is in the form of an aqueous lotion (non-emulsion).

The compositions may be packaged in various forms, especially in a tube, a jar or bottles, in pump bottles, in squeeze bottles, or in aerosol containers so as to apply the composition in vaporized form or in the form of a mousse. The compositions may also impregnate applicators, especially gloves or wipes.

The composition may be applied by hand, with an applicator nozzle or actuator pump, with a container equipped with a pump, an applicator and a dispensing comb, or with an insoluble substrate impregnated with the composition.

As used herein, the process and composition disclosed herein may be used on the hair that has not been artificially dyed, pigmented or permed.

As used herein, the process and composition disclosed herein may be also used on the hair that has been artificially dyed, pigmented or permed, relaxed, straightened or other chemical process.

The compositions according to the disclosure may be prepared according to techniques that are well known to those skilled in the art.

Although the foregoing refers to various exemplary embodiments, it will be understood that the disclosure is not so limited. It will occur to those of ordinary skill in the art that various modifications may be made to the disclosed embodiments and that such modifications are intended to be within the scope of the disclosure. Where an embodiment employing a particular structure and/or configuration is illustrated in the present disclosure, it is understood that the present disclosure may be practiced with any other compatible structures and/or configurations that are functionally equivalent provided that such substitutions are not explicitly forbidden or otherwise known to be impossible to one of ordinary skill in the art.

The following examples are intended to further illustrate the present invention. They are not intended to limit the invention in any way. Unless otherwise indicated, all parts are by weight.

In some embodiments, the invention provides a treatment of a keratinous substrate comprising: at least one polycarbodiimide compound; and at least one cationic polymer selected from polyquatemium cationic polymers, wherein the at least one polycarbodiimide compound and polyquatemium cationic polymer are present in a ratio of about 1:1, wherein the composition comprises from about 0.1 to about 40%, by weight, of the composition of a combined amount of the polycarbodiimide compound and the cationic polymer compound, wherein the composition includes amounts of each of the polycarbodiimide compound and the cationic polymer compound sufficient to impart one or more of increased hydrophobicity to the keratinous substrate after application thereto and increased hold to the shape or configuration of the keratinous substrate.

In some embodiments, each of the polycarbodiimide compound and the polyquatemium cationic polymer is present in a concentration, by weight, of from about 0.01% to about 20%.

In some embodiments, each of the polycarbodiimide compound and the polyquatemium cationic polymer is present in a concentration, by weight, of from about 0.01% to about 10%.

In some embodiments, each of the polycarbodiimide compound and the polyquatemium cationic polymer is present in a concentration, by weight, of from about 1% to about 6%.

9 In some embodiments, the composition includes an additive selected from one or a combination of carboxysilicone polymers, cationic polymer compounds, and latex polymers.

In some embodiments, the cationic polymer is selected from the group consisting of polyquatemium 22, polyquaternium-39, polyquaternium-47, polyquaternium-53); polyquatemium-30, polyquatemium-35, polyquatemium-45, polyquatemium-50, polyquatemium-54; polyquaternium-57; polyquatemium-63; polyquaternium-74; polyquatemium-76; polyquaternium-86; polyquatemium-89; polyquaternium-95; polyquatemium-98, polyquatemium-104; polyquaternium-111; polyquatemium-112, and mixtures thereof.

In some embodiments, the cationic polymer compound is selected from the group consisting of polyquaternium 22, polyquatemium-39, polyquaternium-47, and polyquaternium-53.

In some embodiments, the hydrophobicity imparted to hydrophilic keratinous substrates confers one or more of frizz control at conditions of high humidity, ease of distribution, ease of brushing, and adhesion.

In some embodiments, the hydrophobicity imparted to hydrophilic keratinous substrates confers conditioning, ease of brushing and good adhesion.

In some embodiments, the combination of polycarbodiimide and cationic polymer compounds is present in an amount, by weight, in the range from about 3% to about 4%, and at a ratio of polycarbodiimide and polyquaternium cationic polymer of 1:1, and the cationic polymer compound comprises one or more or of polyquatemium-39, polyquatemium-47, and polyquatemium-53.

In some embodiments, the invention provides a method of protecting a keratinous substrate chosen from hair, eyelashes and eyebrows from extrinsic damage caused by heating, UV radiation, chemical treatment or mechanical stress, or of repairing a keratinous substrate chosen from hair, eyelashes and eyebrows following extrinsic damage caused by heating, UV radiation, chemical treatment, or mechanical stress comprising, wherein each of the polycarbodiimide and the cationic polymer compound of a composition according to claim 1 is provided in a premix comprising at least a solvent, and the premix is applied to the keratinous substrate according to a process selected from a one step process and a two-step process.

In some embodiments, the process is at least a one step process selected from a one step process, wherein the composition comprising the polycarbodiimide and the cationic polymer compound is provided as a premix, and is prepared by combining the polycarbodiimide and cationic polymer compound and at least a solvent, whereby the premixed composition is applied onto the keratinous substrate; a one step process, wherein the composition is provided in separate premixes, each separately comprising the polycarbodiimide and the cationic polymer compound, the premixes prepared by combining the polycarbodiimide with at least one solvent to form on first phase, and separately combining the cationic polymer compound with at least one solvent to form a second phase, whereby at the time of use, the premixed phases are combined and to form a composition that is applied onto the keratinous substrate; and a two-step process, wherein the composition is provided in separate premixes, the premixes prepared by combining the polycarbodiimide with water to form on a first phase, and separately combining the cationic polymer compound with a solvent chosen from one or more of water and an organic solvent to form a second phase, whereby at the time of use, the each of the premixed phases is applied separately to the keratinous substrate in any order.

In some embodiments, the process is a two-step process, comprising the steps of (i) applying the polycarbodiimide phase, then (ii) applying the polyquaternium cationic polymer phase, wherein the process confers greater lasting frizz control under high humidity conditions, and the cationic polymer compound comprises at least polyquaternium-22.

In some embodiments, the invention provides an article of manufacture comprising a kit containing, in separately packaged form, the kit comprising: at least one of: a composition according to claim 1 wherein the polycarbodiimide and cationic polymer compound are combined and the premix comprises at least a solvent; and a composition according to claim 1 wherein the polycarbodiimide and cationic polymer compound are provided in in separate packages, comprising a packaged aqueous phase premix that comprises polycarbodiimide and at least a solvent comprising water; and also comprising a packaged non-aqueous phase premix that comprises cationic polymer compound with at least an organic solvent, whereby at the time of use, the premixed phases are combined and agitated to form an emulsion.

In some embodiments, the kit comprising: at least one of: a separately packaged premix comprising a processing agent selected from a coloring agent, a pigmenting agent, a permanent process agent, a relaxing process agent, a straightening process agent, and a highlighting process agent; and a separately packaged premix comprising at least one of the polycarbodiimide and the cationic polymer compound of a composition according to claim 1, and optionally, at least one processing agent selected from a coloring agent, a pigmenting agent, a permanent process agent, a relaxing process agent, a straightening process agent, and a highlighting process agent.

Procedure for preparation of Hair Treatment Composition (For Hair Repair or Hair Styling)

Stock solutions of each phase were generated by stirring a selected amount or percentage by weight of active RM (raw material) in the selected solvent (either water, or mixture of water and organic solvent such as Isododecane (IDD) or ethanol). Just before application, the desired ratio of parts (typically 1:1 actives) were weighed into a vial and shaken to create the hair treatment solution. Typically, 1 g of product was applied to 1 g of hair. In the case of 2-step treatments, stock solutions were applied directly to hair without pre mixing.

### EXAMPLES

### 1: Examples with Polycarbodiimide and Carboxylic Acid Compound Chosen from Fatty Acids, their Salts, and Mixtures Thereof

The following examples are to illustrate the invention and are non-limiting.

Raw Materials Employed in the Examples

Designations and Ingredient Names

Polycarbodiimide 1
Commercially available as V-02-L2 from the supplier Nisshinbo

A. Testing Procedures

1) Procedure for Durability determination using High humidity curl retention (HHCR) test

Hair Treatment

Regular bleached hair swatch (from IHIP, 13.5 cm long, about 0.5 g weight) was treated with solutions of 4% by weight of active material of test solutions (0.5 g solution/g hair). The hair was combed until the solution was uniformly distributed over the hair swatch surface. The treated hair was then rolled onto a spiral rod (0.5 in diameter) and allowed to:

a) Dry at room temperature overnight or

b) Dry in a 50 C oven for 30 minutes and then dried at room temperature overnight.

Curl Retention Measurement

The coiled hair was removed from the rod and placed in the humidity chamber at 90% RH, 40° C. for 5 hours. % Curl Retention was calculated using the formula below:

$$\% Curl Retention = \frac{( Lo - Lt )}{( Lo - Li )} * 100$$

Where: Lo=Original hair length (fully extended hair length)
Li=Initial hair length (length of hair before humidity exposure)
Lt=Length of hair after 5 hr humidity exposure

Procedure for Hydrophobicity Test Using Contact Angle

Hair Treatment

A strip or swatch of twice-bleached hair (from IHIP, 0.5 cm wide, approx. 0.75 g) was treated with the inventive composition (1 g treatment/g hair) and placed in an oven at 50° C. for 30 minutes, then allowed to dry overnight. The following morning, the hair was washed with DOP shampoo, rinsed, dried in a helmet dryer and measured for residual hydrophobicity.

Hydrophobicity Measurement

Hydrophobicity of each swatch was measured via contact angle measurements using Biolin Scientific Contact Angle Tensiometer, Model C204A. A bundle of 30-50 fibers was clamped to create a flat surface. A 3 L drop of deionized (DI) H2O was placed on the fiber surface and the contact angle was measured for 10 seconds. The values reported below are an average of 3 measurements using the contact angle at 10 seconds.

### B. Examples

I. High Humidity Curl Retention of Hair Treated with Isostearic Acid and Polycarbodiimide 1

Three hair swatches are treated with 4% active treatment (0.5 g of product/g of hair):

1) 4% Polycarbodiimide 1 in water

2) 2% Polycarbodiimide 1+2% Isostearic Acid (neutralized to pH 6 with Ammonium Hydroxide)

3) 4% Isostearic Acid (neutralized to pH 6 with Ammonium Hydroxide) in Water

The hair swatches were then dried overnight around spiral curling rods. The high humidity curl retention results are shown below.

| | % Curl |
|---|---|
| Treatment | Retained after 5 hours |
| 4% Polycarbodiimide 1 | 8.93% |
| 2% Polycarbodiimide 1 + 2% Isostearic Acid | 50.72% |
| 4% Isostearic Acid | 14.93% |

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

Incorporating polycarbodiimide resulted in a coating on the hair with increased humidity resistance properties achieved through crosslinking interactions with the carboxylic acid and with the hair.

II. High Humidity Curl Retention of Hair Treated with Isostearic Acid and Polycarbodiimide 1 with Heat

Three hair swatches were treated with 4% active treatment solutions (0.5 g/g of product):

1) 4% active Polycarbodiimide 1 in water

2) 2% Polycarbodiimide 1+2% Isostearic Acid (neutralized to pH 6 with Ammonium Hydroxide)

3) 4% Isostearic Acid in Water (neutralized to pH 6 with Ammonium Hydroxide)

The hair swatches were then dried in a 50° C. oven for 30 minutes, followed by further drying at room temperature overnight on spiral curling rods. The high humidity curl retention results are shown below.

| Treatment | % Curl Retained after 5 hours |
|---|---|
| 4% Polycarbodiimide 1 | 26.15% |
| 2% Polycarbodiimide 1 + | 51.47% |
| 2% Isostearic Acid | |
| 4% Isostearic Acid | 7.25% |

The results indicate that hair treated with the inventive compositions and dried at 50° C. for 30 minutes and then room temperature has an increased percentage of curl retained indicating higher resistance to high humidity and high temperature compared to hair treated with either material alone. With the use of heat (drying in the oven), the percent curl retained by the use of the inventive composition was greater than the no heat application in Example I. It can be expected that the same or similar performance as obtained in accordance with the invention can be achieved using other types of heating tools such as a blow drier (60° C.), flat iron (120-250° C.), steam/hot rollers and the like.

III. High Humidity Curl Retention of Hair Treated with Sodium Stearate and Polycarbodiimide 1 with Heat

Three hair swatches were treated with 4% active treatment solutions (0.5 g/g of product):

4% Sodium Stearate in Water

2% Sodium Stearate+2% Polycarbodiimide 1

4% Polycarbodiimide 1 in water

The hair was then dried in a 50 C oven for 30 minutes, followed by further drying at room temperature overnight on spiral curling rods. The high humidity curl retention results are shown below.

| Treatment | % Curl Retained after 5 hours |
|---|---|
| 4% Sodium Stearate | 15.38% |
| 2% Polycarbodiimide 1 + | 35.38% |
| 2% Sodium Stearate | |
| 4% Polycarbodiimide 1 | 17.46% |

The results indicate that hair treated with the inventive compositions and dried at 50° C. for 30 minutes and then room temperature has increased in percent curl retained indicating higher resistance to high humidity and high temperature compared to hair treated with either material alone. It can be expected that the same or similar performance as obtained in accordance with the invention can be achieved using other types of heating tools such as a blow drier, flat iron, steam/hot rollers and the like.

The above examples show that cross linking the fatty acids with carbodiimide and hair results in better style and shape memory through resiliency against high humidity.

IV. Hydrophobicity of Hair Treated with Isostearic Acid and Polycarbodiimide 1

Three hair swatches were treated with the following treatment solutions (1 g product/g of hair):

2% Isostearic acid (pH 6, adjusted with ammonia) in Water

2% Ca Polycarbodiimide 1 in water

2% Isostearic Acid (pH 6)+2% Polycarbodiimide 1

The hair was then placed in a 50° C. oven for 30 minutes and dried overnight at room temperature overnight. After shampoo, these swatches exhibited the following hydrophobicity:

| Treatment | Contact Angle |
|---|---|
| 2% Isostearic Acid | 0° |
| 2% Polycarbodiimide 1 | 0° |
| 2% Polycarbodiimide 1 + | 79.8 ± 17.8° |
| 2% Isostearic Acid | |

The above example illustrates that the reaction between fatty acids and poly(carbodiimide) gives hydrophobicity to very hydrophilic hair that is able to survive a shampoo treatment.

V. Hydrophobicity of Hair Treated with Sodium Stearate and Polycarbodiimide 1

Three hair swatches were treated with the following treatment solutions (1 g product/g of hair):

2% Sodium Stearate in Water

2% Polycarbodiimide 1 in water

2% Sodium Stearate+2% Polycarbodiimide 1

The hair was then placed in a 50° C. oven for 30 minutes and dried overnight at room temperature overnight. After shampoo, these swatches exhibited the following hydrophobicity:

| Treatment | Contact Angle |
|---|---|
| 2% Sodium Stearate | 0° |
| 2% Polycarbodiimide 1 | 0° |
| 2% Polycarbodiimide 1 + | 70.7 ± 30.2° |
| 2% Sodium Stearate | |

The above example illustrates that the reaction between fatty acids and poly(carbodiimide) gives hydrophobicity to very hydrophilic hair (damaged hair) that is able to survive a shampoo treatment.

### 2: Examples with Polycarboxylic Acid Compound

Generic Procedure for Preparation of Hair Repair Treatment

Stock solutions of each phase were generated by stirring the active RM in the respective solvent (either water, Isododecane (IDD) or ethanol). Just before application, the desired ratio of parts (typically 1:1 actives) were weighed into vial and shaken vigorously by hand for 10 seconds/g of solution to create milky emulsion. 1 g of product was applied to 1 g of hair. In the case of 2-step treatments, stock solutions were applied directly to hair without pre mixing.

Procedure for Preparation of Hair Styling Composition

4% Stock solutions of each phase were generated by stirring the active RM in the respective solvent (either water or Isododecane (IDD)). Just before application, the desired ratio of parts (typically 1:1 actives) were weighed into vial and shaken vigorously by hand for 10 seconds to create milky emulsion.

The following examples are intended to further illustrate the present invention. They are not intended to limit the invention in any way. Unless otherwise indicated, all parts are by weight.

### EXAMPLES

The following examples are to illustrate the invention and are non-limiting.

The cosmetic application of polycarbodiimide and non-silicone carboxylic acid containing polymer association for hair applications

Testing Procedures

Procedure for Durability determination using High humidity curl retention (HHCR) test

Hair Treatment

Regular bleached hair swatch (from IHIP, 13.5 cm long, about 0.5 g weight) is treated with solutions of 4% by weight of active material of test solutions (0.5 g solution/g hair). The hair is combed until the solution is uniformly distributed over the hair swatch surface. The treated hair is then rolled onto a spiral rod (0.5 in diameter) and allowed to:

Dry at room temperature overnight or

Dry in a 50 C oven for 30 minutes and then dried at room temperature overnight.

Curl Retention Measurement

The coiled hair is removed from the rod and placed in the humidity chamber at 90% RH, 40° C. for 5 hours. % Curl Retention was calculated using the formula below:

$$\% Curl Retention = \frac{( Lo - Lt )}{( Lo - Li )} * 100$$

Where: Lo=Original hair length (fully extended hair length)
Li=Initial hair length (length of hair before humidity exposure)
Lt=Length of hair after 5 hr humidity exposure

Procedure for Determination of the Mechanical Property of Treated Hair Using Three Point Bending

Hair Treatment

A strip of normal hair (from IHIP, 1 cm in width, 15 cm in length, about 2.0-2.5 g of hair) was treated with the testsolution (0.5 g of aqueous solution/g hair). The hair was combed through until the solution was uniformly distributed over the surface of the tress. The treated hair, in a straight configuration, was then allowed to dry overnight at room temperature.

Three-Point Bending Measurement

The test was conducted using a texture analyzer (Model TA-XTPlus, Texture Technologies Corporation) equipped with a hair mounting accessory as described in J. Cosmet. Sci., 53, 345-362 (November/December 2002). The cantilever bending experiment consisted of the following sequence of steps: the hair tress was placed on a 2-point of 6 cm width, and the probe, representing the third point, came down at the middle of the hair tress and performed 10 cycles of 10-mm deformations of the hair tress. The testing protocol was:

Test mode=Compression

Pre-test speed=2 mm/sec

Test speed=2 mm/sec

Post-test speed=2 mm/sec

Target mode=Distance

Distance=10 mm

Count=10

Trigger type=Auto (Force)

Trigger force=1 g

After finishing 10 cycles of bending, a plot of force as a function of distance of 10 deformations was generated. From the plot, the maximum force in the first deformation was determined.

A high maximum force indicates that the hair was stiff with strong hold, and a lower maximum force indicates that the hair was softer with weaker hold.

Each experiment was run three times, and the results are reported from the average of the three experiments.

Procedure for Hydrophobicity Test using Contact Angle

Hair Treatment

A strip of twice-bleached hair (from IHIP, 0.5 cm wide, approx. 0.75 g) was treated with the test composition (1 g treatment/g hair) and placed in an oven at 50° C. for 30 minutes, then allowed to dry overnight. The following morning, the hair was washed with a conventional sulfate-based shampoo (DOP shampoo), rinsed, dried in a helmet dryer and measured for residual hydrophobicity.

Hydrophobicity Measurement

Hydrophobicity of each swatch was measured via contact angle measurements using Biolin Scientific Contact Angle Tensiometer, Model C204A. A bundle of 30-50 fibers was clamped to create a flat surface. A 3-5 uL drop of DI H₂O was placed on the fiber surface and the contact angle was measured for 10 seconds. The values reported below are an average of 3 measurements using the contact angle at 10 seconds.

Sufficiently porous hair or hydrophilic hair will not support a droplet for a full 10 seconds. In these cases, the time for the droplet to fully absorb/spread is instead reported.

Raw Materials Employed in the Examples

Designations and Ingredient Names

PEMULEN TR 2 (“Pemulen TR 2” or “Pemulen”)
(acrylic acid/C10-C30 alkyl acrylate crosslinked copolymer)
AMPHOMER LV 71 (“Amphomer”)
(octylacrylamide/acrylates/butylaminoethyl methacrylate copolymer)
RESYN 28-29-30 (or “Resyn”)
(crotonic acid/vinyl acetate/vinyl neododecanoate terpolymers)
LICOCARE CM 401 LP 3345 (or “Clariant CM 401”)
alpha olefin hydrocarbon - maleic anhydride copolymer wax
Polycarbodiimide (“Carbodilite V02-L2” or “Carbodilite”))
Commercially available as CARBODILITE V-02-L2 from the supplier
Nisshinbo

High Humidity Curl Retention of Hair Treated with Pemulen TR 2 and Carbodilite V02-L2

Three hair swatches are treated (0.5 g/g of product):

4% active Carbodilite V02-L2 in water

0.2% Pemulen TR-2 neutralized with ammonia to pH 6.5-7 and dissolved in water to form a clear solution

2% Carbodilite V02-L2+0.1% Pemulen TR-2 (neutralized with ammonia to pH 6.5-7 and dissolved in water).

The hair was then dried overnight around the curling rods. The high humidity curl retention results are shown below.

| Treatment | % Curl Retained after 5 hours |
|---|---|
| 4% V-02 L2 | 13.04% |
| 2% V02-L2 + 0.1% | 60.87% |
| Pemulen TR-2 | |
| 0.2% Pemulen TR-2 | 38.46% |

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity. Incorporating Carbodilite into the coating increases the humidity resistance through crosslinking.

High Humidity Curl Retention of Hair Treated with Pemulen TR 2 and Carbodilite V02-L2 with Heat

Three hair swatches are treated (0.5 g/g of product):

4% active Carbodilite V02-L2 in water
0.2% Pemulen TR-2 (53971) neutralized with ammonia to pH 6.5-7 and dissolved in water to form a clear solution

The hair was then dried in a 50 C oven for 30 minutes, followed by further drying at room temperature overnight on spiral curling rods. The high humidity curl retention results are shown below.

| | % Curl |
|---|---|
| Treatment | Retained after 5 hours |
| 4% V02-L2 | 17.46% |
| 2% V02-L2 + 0.1% Pemulen TR-2 | 78.26% |
| 0.2% Pemulen TR-2 | 22.39% |
| John Frieda Frizz Ease Extra | 2.38% |
| Strength Serum | |
| Tresseme Mousse Flawless Curl | 17.39% |

The results indicate that hair treated with the inventive compositions and dried at 50 C for 30 minutes and then room temperature has increased in percent curl retained indicating higher resistance to high humidity and high temperature compared to hair treated with either material alone. With the use of heat (drying in the oven), the percent curl retained of the inventive composition was greater than the no heat application this indicates superior performance with commonly used heat tools such as a blow drier (60 C), flat iron (120 C-250 C).

Additionally the inventive composition preforms better than two leading industry benchmarks that claim humidity resistance.

High Humidity Curl Retention of Hair Treated with Maleic Anhydride (Licocare CM 401) and Carbodilite V02-L2 with Heat

Three hair swatches are treated (0.5 g/g of product):

4% active Carbodilite V02-L2 in water

4% MA

2% Carbodilite V02-L2+2% MA

The hair was then dried in a 50 C oven for 30 minutes, followed by further drying at room temperature overnight on spiral curling rods. The high humidity curl retention results are shown below.

| Treatment | % Curl Retained after 5 hours |
|---|---|
| 4% V-02 L2 | 17.46% |
| 2% V02-L2 + 2% MA | 55.07% |
| 4% MA | 3.33% |
| John Frieda Frizz Ease Extra | 2.38% |
| Strength Serum | |
| Tresseme Mousse Flawless Curl | 17.39% |

The results indicate that hair treated with the inventive compositions and dried at 50 C for 30 minutes and then room temperature has increased in percent curl retained indicating higher resistance to high humidity and high temperature compared to hair treated with either material alone. With the use of heat (drying in the oven), the percent curl retained of the inventive composition was greater than the no heat application this indicates superior performance with commonly used heat tools such as a blow drier (60 C), flat iron (120 C-250 C).

Additionally the inventive composition preforms better than two leading industry benchmarks that claim humidity resistance.

Mechanical Property of Hair Treated with Pemulen TR 2 and Carbodilite V02-L2

Normal virgin hair swatches (about 2.0-2.5 g) were treated with the following aqueous solutions (0.5 g product/g hair).

4% active Carbodilite V02-L2 in water
0.2% Pemulen TR-2 neutralized with ammonia to pH 6.5-7 and dissolved in water to form a clear solution
2% Carbodilite V02-L2+0.1% Pemulen TR-2 (neutralized with ammonia to pH 6.5-7 and dissolved in water)

The treated hair swatches were dried at room temperature overnight. A 3-point bending test was performed on these hair swatches (replicates of 3) to determine the maximum force (FMax) to bend the hair 10 cm downward.

| Treatment | Maximum Force (g) |
|---|---|
| Pemulen (0.2%) | 67 |
| V02-L2 (4a) | 116 |
| V02-L2 (2%) + Pemulen (0.1%) | 432 |

The results indicate that hair treated with the inventive compositions has increased in stiffness, almost 4 times stronger hold compared to hair treated with either material alone. Incorporating Carbodilite into the coating increases the stiffness through crosslinking. This increased film stiffness translates to improved shaping performance of the coating.

The above examples show that cross linking the non-silicone carboxylated polymer with carbodiimide results in higher stiffness/rigidity, style and shape memory through resiliency against high humidity.

Hydrophobicity of Twice-Bleached Hair Treated with Carbodilite V-02 L2 and Clariant CM401

Twice bleached hair swatches (about 0.75 g) were treated with the following aqueous solutions (1 g product/g hair).

2% Carbodilite V-02 L2 in 50:50 water/isododecane mix

2% hydrolyzed Clariant CM401 in a 50:50 water/isododecane mix

2% Carbodilite V02-L2+2% hydrolyzed Clariant CM401 in a 50:50 water/isododecane mix

After treatment, heating and dry, the swatches were washed, dried again and residual hydrophobicity was measured:

| Treatment | Water droplet absorption time (s) |
|---|---|
| V-02 L2 (2%) | 0.00 |
| CM-401 (2%) | 0.00 |
| V-02 L2 (2%) + CM-401 (2%) | 5.36 |

The above example shows the robustness of the adhesion of this covalent complex as the benefit (increased hydrophobicity) survives shampoo.

Examples Using Testing Method 1

High Humidity Curl Retention of Hair Treated with Resyn and Carbodilite V02-L2

Hair swatches were treated with 4% active solutions (0.5 g/g hair) of the following compositions:

4% active Carbodilite V02-L2 in water

4% Resyn in Ethanol

2% Carbodilite V02-L2+2% Resyn (Shaken before application)

The hair was then dried overnight around the curling rods. The high humidity curl retention results are shown below.

| Treatment | % Curl Retained after 5 hours |
|---|---|
| 4% V-02 L2 | 8.93% |
| 2% V02-L2 + 2% Resyn | 81.43% |
| 4% Resyn | 14.29% |

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity. Incorporating Carbodilite into the coating increases the humidity resistance through crosslinking.

High Humidity Curl Retention of Hair Treated with Resyn (52375) and Carbodilite V02-L2 with Heat

Hair swatches were treated with 4% active solutions (0.5 g/g hair) of the following compositions:

4% active Carbodilite V02-L2 in water

4% Resyn in Ethanol

2% Carbodilite V02-L2+2% Resyn (Shaken before application)

The hair was then dried in a 50 C oven for 30 minutes, followed by further drying at room temperature overnight on spiral curling rods. The high humidity curl retention results are shown below.

| Treatment | % Curl Retained after 5 hours |
|---|---|
| 4% V-02 L2 | 26.15% |
| 2% V02-L2 + 2% Resyn | 95.52% |
| 4% Resyn | 22.22% |

The results indicate that hair treated with the inventive compositions and dried at 50 C for 30 minutes and then room temperature has increased in percent curl retained indicating higher resistance to high humidity and high temperature compared to hair treated with either material alone. With the use of heat (drying in the oven), the percent curl retained of the inventive composition was greater than the no heat application this indicates superior performance with commonly used heat tools such as a blow drier (60 C), flat iron (120 C-250 C).

High Humidity Curl Retention of Hair Treated with Amphomer (52240) and Carbodilite V02-L2

Hair swatches were treated with 4% active solutions (0.5 g/g hair) of the following compositions:

4% active Carbodilite V02-L2 in water

4% Amphomer in Ethanol

2% Carbodilite V02-L2+2% Amphomer (Shaken before application)

The hair was then dried overnight around the curling rods. The high humidity curl retention results are shown below.

| | % Curl Retained |
|---|---|
| Treatment | after 5 hours |
| 4% V-02 L2 | 8.93% |
| 2% V02-L2 + 2% Amphomer | 87.32% |
| 4% Amphomer | 77.33% |

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity. Incorporating Carbodilite into the coating increases the humidity resistance through crosslinking.

High Humidity Curl Retention of Hair Treated with Amphomer and Carbodilite V02-L2 with Heat

Hair swatches were treated with 4% active solutions (0.5 g/g hair) of the following compositions:

4% Carbodilite V02-L2 in water

4% Amphomer in Ethanol

2% Carbodilite V02-L2+2% Amphomer (Shaken before application)

The hair was then dried in a 50 C oven for 30 minutes, followed by further drying at room temperature overnight on spiral curling rods. The high humidity curl retention results are shown below.

| | % Curl Retained |
|---|---|
| Treatment | after 5 hours |
| 4% V-02 L2 | 15% |
| 2% V02-L2 + 2% Amphomer | 90% |
| 4% Amphomer | 61.11% |

The results indicate that hair treated with the inventive compositions and dried at 50 C for 30 minutes and then room temperature has increased in percent curl retained indicating higher resistance to high humidity and high temperature compared to hair treated with either material alone. With the use of heat (drying in the oven), the percent curl retained of the inventive composition was greater than the no heat application this indicates superior performance with commonly used heat tools such as a blow drier (60 C), flat iron (120 C-250 C).

New Testing Method 4

Procedure for Shampoo Resistance determination using High humidity curl retention (HHCR) test

Hair Treatment

Wash Hair swatches from Testing Method 1 (Apply product (allow to dry with or without heat, run high humidity curl retention at 90% humidity 40 C). Remove swatches from humidity chamber and allow to sit at ambient room temperature.

Washing Treatment

Using commercially available shampoo.

Wash hair with 0.4 g of shampoo/g hair. Massage hair for 10 seconds with shampoo and rinse with water for 20 seconds. Comb the hair twice to detangle hair. Wrap the hair swatch around the curling rod. Allow to dry overnight at room temperature.

Curl Retention Measurement

The coiled hair is removed from the rod and placed in the humidity chamber at 90% RH, 40° C. for 5 hours. % Curl Retention was calculated using the formula below:

$$\% Curl Retention = \frac{( Lo - Lt )}{( Lo - Li )} * 100$$

Where: Lo=Original hair length (fully extended hair length)
Li=Initial hair length (length of hair before humidity exposure)
Lt=Length of hair after 5 hr humidity exposure

The hair was then dried overnight around the curling rods after 1 shampoo. The high humidity curl retention results are shown below.

| Treatment | % Curl Retained after 5 hours |
|---|---|
| 4% V-02 L2 | 14.29% |
| 2% V02-L2 + 2% Resyn | 28.57% |
| 4% Resyn | 7.58% |

The results indicate that hair treated with the inventive compositions at room temperature has increased percent curl retained indicating higher resistance to shampoo, high humidity and high temperature compared to hair treated with either material alone. The inventive composition displayed a higher styling property over time and through shampoo treatment at high humidity condition as indicated by the higher curl retention value. In contrast, the hair treated with either material alone demonstrated a lower curl retention value, indicating less styling hold over time and through shampoo at high humidity. Incorporating Carbodilite into the coating increases the wash resistance and humidity resistance through crosslinking.

These swatches were originally dried in a 50 C oven for 30 minutes and then dried at room temperature after 1 shampoo. No additional heat was used to dry once the swatches were washed.

| | % Curl Retained |
|---|---|
| Treatment | after 5 hours |
| 4% V-02 L2 (with 30 mins heat) | 13.89% |
| 2% V02-L2 + 2% Resyn (with 30 mins heat) | 6.67% |
| 4% Resyn (with 30 mins heat) | 23.08% |

The results indicate that hair treated with the inventive compositions at room temperature has increased percent curl retained indicating higher resistance to shampoo, high humidity and high temperature compared to hair treated with either material alone. The inventive composition displayed a higher styling property over time and through shampoo treatment at high humidity condition as indicated by the higher curl retention value. In contrast, the hair treated with either material alone demonstrated a lower curl retention value, indicating less styling hold over time and through shampoo at high humidity. Incorporating Carbodilite into the coating increases the wash resistance and humidity resistance through crosslinking with and without heat.

3: Examples with Cationic Polymer

### Example I

The following examples are to illustrate the invention and are non-limiting.

Testing Procedures

Procedure for Durability determination using High humidity curl retention (HHCR) test.

Hair Treatment

Regular bleached hair swatch (from IHIP, 13.5 cm long, about 0.5 g weight) is treated with solutions of 4% by weight of active material of test solutions (0.5 g solution/g hair). The hair is combed until the solution is uniformly distributed over the hair swatch surface. The treated hair is then rolled onto a spiral rod (0.5 in diameter) and allowed to:

Dry at room temperature overnight or

Dry in a 50° C. oven for 30 minutes and then dried at room temperature overnight.

Curl Retention Measurement

The coiled hair is removed from the rod and placed in the humidity chamber at 90% RH, 40° C. for 5 hours. % Curl Retention was calculated using the formula below:

$$\% Curl Retention = \frac{( Lo - Lt )}{( Lo - Li )} * 100$$

Where: Lo=Original hair length (fully extended hair length)

Li=Initial hair length (length of hair before humidity exposure)

Lt=Length of hair after 5 hr. humidity exposure

Procedure for Determination of the mechanical property of treated hair using Three Point Bending

Hair Treatment

A strip of normal hair (from IHIP, 1 cm in width, 15 cm in length, about 2.0-2.5 g of hair) was treated with the tested solution (0.5 g of aqueous solution/g hair). The hair was combed through until the solution was uniformly distributed over the surface of the tress. The treated hair, in a straight configuration, was then allowed to dry overnight at room temperature.

Three-Point Bending Measurement

The test was conducted using a texture analyzer (Model TA-XTPlus, Texture Technologies Corporation) equipped with a hair mounting accessory as described in J. Cosmet. Sci., 53, 345-362 (November/December 2002). The cantilever bending experiment consisted of the following sequence of steps: the hair tress was placed on a 2-point of 6 cm width, and the probe, representing the third point, came down at the middle of the hair tress and performed 10 cycles of 10-mm deformations of the hair tress. The testing protocol was:

Test mode=Compression
Pre-test speed=2 mm/sec
Test speed=2 mm/sec
Post-test speed=2 mm/sec
Target mode=Distance

### Distance=10 mm

### Count=10

Trigger type=Auto (Force)
Trigger force=1 g

After finishing 10 cycles of bending, a plot of force as a function of distance of 10 deformations was generated. From the plot, the maximum force in the first deformation was determined.

A high maximum force indicates that the hair was stiff with strong hold, and a lower maximum force indicates that the hair was softer with weaker hold.

Each experiment was run three times, and the results are reported from the average of the three experiments.

Procedure for Hydrophobicity Test using Contact Angle

Hair Treatment

A strip of twice-bleached hair (from IHIP, 0.5 cm wide, 0.75 g) was treated with aqueous solutions of the inventive composition. These swatches were dried in an over at 50° C. for 30 minutes, then allowed to dry overnight. The following morning, swatches were washed with DOP shampoo, dried in a helmet dryer and measured for residual hydrophobicity.

Hydrophobicity Measurement

Hydrophobicity of each swatch was measured via contact angle measurements using Biolin Scientific Contact Angle Tensiometer, Model C204A. A bundle of 30-50 fibers was clamped to create a flat surface. A 3-5 uL drop of DI H₂O was placed on the fiber surface and the contact angle was measured for 10 seconds. The values reported below are an average of 3 measurements using the contact angle at 10 seconds.

High Humidity Curl Retention of Hair Treated with Polyquaternium-22 (PQ-22) and Carbodilite V02-L2

Three hair swatches are treated with 4% active solutions (0.5 g/g of product), as shown below.

TABLE 1

4% Carbodilite V02-L2
2% Carbodilite V02-L2 + 2% PQ 22
4% PQ 22

The hair was then dried overnight around the curling rods. The high humidity curl retention results are shown below.

TABLE 2

| Treatment | % Curl Retained after 5 hours |
|---|---|
| 4% V02-L2 | 13.04% |
| 2% V02-L2 + 2% PQ 22 | 30.43% |
| 4% PQ 22 | 21.73% |

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

High Humidity Curl Retention of Hair Treated with Polyquaternium-47 and Carbodilite V02-L2

Three hair swatches are treated with 4% active solutions (0.5 g/g of product):

TABLE 3

4% Carbodilite V02-L2
2% Carbodilite V02-L2 + 2% PQ 47
4% PQ 47

The hair was then dried overnight around the curling rods. The high humidity curl retention results are shown below.

TABLE 4

| Treatment | % Curl Retained after 5 hours |
|---|---|
| 4% V02-L2 | 13.04% |
| 2% Carbodilite V02-L2 + 2% PQ 47 | 30.43% |
| 4% PQ 47 | 17.14% |

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

High Humidity Curl Retention of Hair Treated with Polyquaternium-22 and Carbodilite V02-L2 with Heat

Three hair swatches are treated with 4% active solutions (0.5 g/g of product):

TABLE 5

4% Carbodilite V02-L2
2% Carbodilite V02-L2 + 2% PQ 22
4% PQ 22

The hair was then dried in a 50° C. oven for 30 minutes, followed by further drying at room temperature overnight on spiral curling rods. The high humidity curl retention results are shown below.

TABLE 6

| | % Curl Retained |
|---|---|
| Treatment | after 5 hours |
| 4% V02 L2 | 17.46% |
| 2% V02 L2 + 2% PQ 22 | 45.71% |
| 4% PQ 22 | 17.64% |
| John Frieda Frizz Ease Extra Strength Serum | 2.38% |
| Tresseme Mousse Flawless Curl | 17.39% |

The results indicate that hair treated with the inventive compositions and dried at 50° C. for 30 minutes and then room temperature has increased in percent curl retained indicating higher resistance to high humidity and high temperature compared to hair treated with either material alone. With the use of heat (drying in the oven), the percent curl retained of the inventive composition was greater than the no heat application this indicates superior performance with commonly used heat tools such as a blow drier (60° C.), flat iron (120° C.-250° C.).

Additionally, the inventive composition performs better than two leading industry benchmarks that claim humidity resistance.

High Humidity Curl Retention of Hair Treated with Polyquaternium-47 and Carbodilite V02-L2 with Heat.

Three hair swatches are treated with 4% active solutions (0.5 g/g of product):

TABLE 7

4% Carbodilite V02-L2
2% Carbodilite V02-L2 + 2% PQ 47
4% PQ 47

The hair was then dried in a 50° C. oven for 30 minutes, followed by further drying at room temperature overnight on spiral curling rods. The high humidity curl retention results are shown below.

TABLE 8

| | % Curl Retained |
|---|---|
| Treatment | after 5 hours |
| 4% V02 L2 | 17.46% |
| 2% V02 L2 + 2% PQ 47 | 58.33% |
| 4% PQ 47 | 17.91% |
| John Frieda Frizz Ease Extra Strength Serum | 2.38% |
| Tresseme Mousse Flawless Curl | 17.39% |

The results indicate that hair treated with the inventive compositions and dried at 50° C. for 30 minutes and then room temperature has increased in percent curl retained indicating higher resistance to high humidity and high temperature compared to hair treated with either material alone. With the use of heat (drying in the oven), the percent curl retained of the inventive composition was greater than the no heat application this indicates superior performance with commonly used heat tools such as a blow drier (60° C.), flat iron (120° C.-250° C.).

Additionally, the inventive composition performs better than two leading industry benchmarks that claim humidity resistance.

Mechanical Property of Hair Treated with Polyquaternium-22 and Carbodilite V02-L2.

Normal virgin hair swatches (about 2.0-2.5 g) were treated with the following aqueous solutions (0.5 g product/g hair).

TABLE 9

2% Carbodilite V02-L2
2% PQ 22
2% Carbodilite V02-L2 + 2% PQ 22

The treated hair swatches were dried at room temperature overnight. A 3-point bending test was performed on these hair swatches (replicates of 3) to determine the maximum force (FMax) to bend the hair 10 cm downward.

TABLE 10

| Treatment | Maximum Force (g) |
|---|---|
| 2% V02 L2 | 60 |
| 2% PQ 22 | 238 |
| 2% V02 L2 + 2% PQ 22 | 395 |

The results indicate that hair treated with the inventive compositions has increased in stiffness, compared to hair treated with either material alone or either material added separately. Incorporating Carbodilite into the coating increases the stiffness through crosslinking of the polymers and the hair. This increased film stiffness translates to improved shaping performance of the coating.

Mechanical Property of Hair Treated with Polyquaternium-47 and Carbodilite V02-L2.

Normal virgin hair swatches (about 2.0-2.5 g) were treated with the following aqueous solutions (0.5 g product/g hair).

TABLE 11

2% Carbodilite V02-L2
2% PQ 47
2% Carbodilite V02-L2 + 2% PQ 47

The treated hair swatches were dried at room temperature overnight. A 3-point bending test was performed on these hair swatches (replicates of 3) to determine the maximum force (FMax) to bend the hair 10 cm downward.

TABLE 12

| Treatment | Maximum Force (g) |
|---|---|
| 2% V02 L2 | 60 |
| 2% PQ 47 | 213 |
| 2% V02 L2 + 2% PQ 47 | 474 |

The results indicate that hair treated with the inventive compositions has increased in stiffness, compared to hair treated with either material alone or either material added separately. Incorporating Carbodilite into the coating increases the stiffness through crosslinking of the polymers and the hair. This increased film stiffness translates to improved shaping performance of the coating.

The above examples show that cross linking polyquat containing carboxylic acid polymer with carbodiimide and the keratin substrate results in higher stiffness/rigidity, style and shape memory through resiliency against high humidity.

Hydrophobicity of Polyquaternium-22

Twice bleached hair swatches (about 0.75 g) were treated with the following aqueous soutions (1 g product/g hair).

TABLE 13

2% Carbodilite V02-L2
2% PQ 22
2% Carbodilite V02-L2 + 2% PQ 22

After shampooing and drying, the following contact angles were measured:

TABLE 14

| Treatment | Water Contact Angle (°) |
|---|---|
| 2% V02 L2 | 0.00 |
| 2% PQ 22 | 38.0 |
| 2% V02 L2 + 2% PQ 22 | 79.33 |

Hydrophobicity of Polyquaternium-53

Twice bleached hair swatches (about 0.75 g) were treated with the following aqueous solutions (1 g product/g hair).

TABLE 15

2% Carbodilite V02-L2
2% PQ 22
2% Carbodilite V02-L2 + 2% PQ 22

After shampooing and drying, the following contact angles were measured:

TABLE 16

| Treatment | Water Contact Angle (°) |
|---|---|
| 2% V02 L2 | 0.00 |
| 2% PQ 53 | 32.0 |
| 2% V02 L2 + 2% PQ 53 | 83.0 |

The above two examples indicate that crosslinking the PQ film on the hair gives the benefits of the PQ treatment a shampoo durability the PQ alone does not possess.

Procedure for Shampoo Resistance Determination Using High Humidity Curl Retention (HHCR) Test

Hair Treatment

Wash Hair swatches from Testing Method 1 (Apply product (allow to dry with or without heat, run high humidity curl retention at 90% humidity 40 C). Remove swatches from humidity chamber and allow to sit at ambient room temperature.

Washing Treatment

Using commercially available shampoo.

Wash hair with 0.4 g of shampoo/g hair. Massage hair for 10 seconds with shampoo and rinse with water for 20 seconds. Comb the hair twice to detangle hair. Wrap the hair swatch around the curling rod. Allow to dry overnight at room temperature.

Curl Retention Measurement

The coiled hair is removed from the rod and placed in the humidity chamber at 90% RH, 40° C. for 5 hours. % Curl Retention was calculated using the formula below:

$$\% Curl Retention = \frac{( Lo - Lt )}{( Lo - Li )} * 100$$

Where: Lo=Original hair length (fully extended hair length)

Li=Initial hair length (length of hair before humidity exposure)

Lt=Length of hair after 5 hr humidity exposure

Shampoo Resistance Through High Humidity of Polyquaternium-47

The hair was then dried overnight around the curling rods after 1 shampoo. The high humidity curl retention results are shown below.

TABLE 17

| Treatment | % Curl Retained after 5 hours |
|---|---|
| 4% V-02 L2 | 14.29% |
| 2% V02-L2 + 2% PQ 47 | 41.67% |
| 4% PQ47 | 7.35% |

The results indicate that hair treated with the inventive compositions at room temperature has increased percent curl retained indicating higher resistance to shampoo, high humidity and high temperature compared to hair treated with either material alone. The inventive composition displayed a higher styling property over time and through shampoo treatment at high humidity condition as indicated by the higher curl retention value. In contrast, the hair treated with either material alone demonstrated a lower curl retention value, indicating less styling hold over time and through shampoo at high humidity. Incorporating Carbodilite into the coating increases the wash resistance and humidity resistance through crosslinking.

Shampoo Resistance Through High Humidity of Polyquaternium-47 (with Heat)

These swatches were originally dried in a 50 C oven for 30 minutes and then dried at room temperature after 1 shampoo. No additional heat was used to dry once the swatches were washed.

TABLE 18

| | % |
|---|---|
| Treatment | Curl Retained after 5 hours |
| 4% V-02 L2 (with 30 mins heat) | 13.89% |
| 2% V02-L2 + 2% PQ47 (with 30 mins heat) | 57.38% |
| 4% PQ47 (with 30 mins heat) | 15.15% |

The results indicate that hair treated with the inventive compositions at room temperature has increased percent curl retained indicating higher resistance to shampoo, high humidity and high temperature compared to hair treated with either material alone. The inventive composition displayed a higher styling property over time and through shampoo treatment at high humidity condition as indicated by the higher curl retention value. In contrast, the hair treated with either material alone demonstrated a lower curl retention value, indicating less styling hold over time and through shampoo at high humidity. Incorporating Carbodilite into the coating increases the wash resistance and humidity resistance through crosslinking with and without heat.

Shampoo Resistance Through High Humidity of Polyquaternium-22 (with Heat)

These swatches were originally dried in a 50 C oven for 30 minutes and then dried at room temperature after 1 shampoo. No additional heat was used to dry once the swatches were washed.

TABLE 19

| | % |
|---|---|
| Treatment | Curl Retained after 5 hours |
| 4% V-02 L2 (with 30 mins heat) | 14.29% |
| 2% V02-L2 + 2% PQ 22 (with 30 mins heat) | 31.88% |
| 4% PQ22 (with 30 mins heat) | 16.67% |

The results indicate that hair treated with the inventive compositions at room temperature has increased percent curl retained indicating higher resistance to shampoo, high humidity and high temperature compared to hair treated with either material alone. The inventive composition displayed a higher styling property over time and through shampoo treatment at high humidity condition as indicated by the higher curl retention value. In contrast, the hair treated with either material alone demonstrated a lower curl retention value, indicating less styling hold over time and through shampoo at high humidity. Incorporating Carbodilite into the coating increases the wash resistance and humidity resistance through crosslinking.

Shampoo Resistant Hydrophobicity of Polyquaternium-22

Twice bleached hair swatches (about 0.75 g) were treated with the following aqueous solutions (1 g product/g hair).

TABLE 20

2% Carbodilite V02-L2
2% PQ 22
2% Carbodilite V02-L2 + 2% PQ 22

These samples were washed once with DOP shampoo to stop the reaction, then dried. They were then washed five additional times with a commercially available shampoo, then dried. After fully drying overnight, the following contact angles were measured.

TABLE 21

| Treatment | Water Contact Angle (°) |
|---|---|
| 2% V02 L2 | 0.00° |
| 2% PQ 22 | 0.00° |
| 2% V02 L2 + 2% PQ 22 | 34.68° |

Shampoo Resistant Hydrophobicity of Polyquaternium-53

Twice bleached hair swatches (about 0.75 g) were treated with the following aqueous solutions (1 g product/g hair).

TABLE 22

2% Carbodilite V02-L2
2% PQ 53
2% Carbodilite V02-L2 + 2% PQ 53

These samples were washed once with DOP shampoo to stop the reaction, then dried. They were then washed five additional times with a commercially available shampoo, then dried. After fully drying overnight, the following contact angles were measured.

TABLE 23

| Treatment | Water Contact Angle (°) |
|---|---|
| 2% V02 L2 | 0.00 |
| 2% PQ 53 | 0.00 |
| 2% V02 L2 + 2% PQ 53 | 73.37 |

The above two examples indicate that crosslinking the PQ film on the hair gives significant shampoo resistance to the coating.

### Example II

The cosmetic application of polycarbodiimide and polyquaternium containing carboxylic acid association for hair applications.

1a) Frizz Control Test and Ease of Brushing for Blowdry Application (with Heat)

Hair swatches (IHIP Frizzy hair) were washed with DOP shampoo containing mainly sodium laureth sulfate, coco-betaine and water. Each of the products in the table below were applied onto designated hair swatches (0.1 g of product/g of hair). The swatches were blow dried (rough dried with blowdrier for 30 seconds to remove excess moisture, then blowdried for 1 minute). Ease of brushing was evaluated.

The hair swatches were then placed in a humidity chamber for 5 hours (90% RH+40° C.). Frizz control was ranked from a scale of 1-5, with 5 corresponding to highest control and least amount of frizz and 1 corresponding to the lowest control and highest amount of frizz.

TABLE 24

| | | Frizz Control |
|---|---|---|
| | Ease of | after hours |
| Treatments* | Brushing | 90% RH 40° C. |
| 3% by weight polyquaternium-53 | 5 | 2 |
| 3% by weight 1:1 polyquaternium 53 + | 5 | 5 |
| polycarbodiimide V02L2 | | |
| 3% by weight polycarbodiimide V02L2 | 1 | 2 |

*amounts of the ingredients in the treatment solutions are expressed in percent total weight of active material in water and ratios are weight ratios

The results indicate that hair treated with the inventive compositions had increased or better properties with respect to ease of brushing and lasting frizz control when exposed to high humidity conditions as compared to hair treated with either material alone. Incorporating polycarbodiimide into a solution containing polyquaternium-53 resulted in increased lastingness of the performance in high humidity conditions.

1b) Frizz Control Test and Ease of Distribution for Air-Dry Application

Hair swatches (IHIP Frizzy hair) were washed with DOP shampoo. Each of the products in the table below were applied onto designated hair swatches (0.05 g/g of hair). Ease of distribution of the solution on the swatch was assessed. The swatches were allowed to dry overnight and were then placed in a humidity chamber for 5 hours (90% RH+40° C.). Frizz control was ranked from a scale of 1-5, with 5 corresponding to highest control and least amount of frizz and 1 corresponding to the lowest control and highest amount of frizz.

TABLE 25

| | | Frizz Control |
|---|---|---|
| | Ease of | after hours |
| Treatments* | Distribution | 90% RH 40° C. |
| 3% by weight polyquaternium-53 | 5 | 2 |
| 3% by weight 1:1 polyquaternium 53 + | 5 | 5 |
| polycarbodiimide V02L2 | | |
| 3% by weight polycarbodiimide | 1 | 3 |
| V02L2 | | |

*amounts of the ingredients in the treatment solutions are expressed in percent total weight of active material in water and ratios are weight ratios

The results indicate that hair treated with the inventive compositions had increased or better properties with respect to ease of distribution when exposed to high humidity conditions as compared to hair treated with either material alone. Incorporating polycarbodiimide into a solution containing polyquaternium-53 resulted in increased lastingness of the performance in high humidity conditions.

2a) Frizz Control Test and Ease of Brushing for Blow-Dry Application (with Heat)

Hair swatches (IHIP Frizzy hair) were washed with DOP shampoo containing mainly sodium laureth sulfate, coco-betaine and water. Each of the products in the table below were applied onto designated hair swatches (0.1 g of product/g of hair). The swatches were blow dried (rough dried with blow-dryer for 30 seconds to remove excess moisture, then blow-dried for 1 minute). Ease of brushing was evaluated.

The hair swatches were then placed in a humidity chamber for 5 hours (90% RH+40° C.). Frizz control was ranked from a scale of 1-5, with 5 corresponding to highest control and least amount of frizz and 1 corresponding to the lowest control and highest amount of frizz.

TABLE 26

| | | Frizz Control |
|---|---|---|
| | Ease of | after 5 hours |
| Treatments* | Brushing | 90% RH 40° C. |
| 3% by weight polyquaternium-39 | 5 | 2 |
| 3% by weight 1:1 polyquaternium 39 + | 5 | 5 |
| polycarbodiimide V02L2 | | |
| 3% by weight polycarbodiimide | 1 | 2 |
| V02L2 | | |

*amounts of the ingredients in the treatment solutions are expressed in percent total weight of active material in water and ratios are weight ratios

The results indicate that hair treated with the inventive compositions had increased or better properties with respect to ease of brushing and lasting frizz control when exposed to high humidity conditions as compared to hair treated with either material alone. Incorporating polycarbodiimide into a solution containing polyquaternium-39 resulted in increased lastingness of the performance in high humidity conditions.

3) Frizz Control Test and Ease of Distribution for Air-Dry Application

Wash hair (IHIP Frizzy Hair) with DOP. Apply solutions (0.05 g/g of hair). Ease of distribution of the solution on the swatch is assessed. Allow to dry overnight. Place in humidity chamber. Frizz control is ranked from a scale of 1-5, with 5 being the highest control and least frizz and 1 being the lowest control and most frizz.

TABLE 27

| | | Frizz Control |
|---|---|---|
| | Ease of | after hours |
| Treatments* | Distribution | 90% RH 40° C. |
| 3% by weight polyquaternium-47 | 5 | 2 |
| 3% by weight 1:1 polyquaternium 47 + | 5 | 5 |
| polycarbodiimide V02L2 | | |
| 3% by weight polycarbodiimide V02L2 | 1 | 3 |

*amounts of the ingredients in the treatment solutions are expressed in percent total weight of active material in water and ratios are weight ratios

The results indicate that hair treated with the inventive compositions had increased or better properties with respect to ease of distribution when exposed to high humidity conditions as compared to hair treated with either material alone. Incorporating polycarbodiimide into a solution containing polyquaternium-47 resulted in increased lastingness of the performance in high humidity conditions.

4) Adhesion+Conditioning Testing of Latex with Carbodilite V02-L2

4% active by weight solutions were poured into polypropylene weighboats (similar surface energy to that of hair). When the resulting films were peeled, the adhesion value was assessed based on the difficulty of peeling the films (with 1 being the easiest and 5 being the hardest to peel). Conditioning is defined as the ease of passing a brush through the treated hair. Hair is treated with 0.25 g of product/g of hair.

TABLE 28

| | | Conditioning/ |
|---|---|---|
| | Adhesion (in | Ease |
| Treatments* | weigh boat) | of Brushing |
| 4% by weight polyquaternium-53 | 1 | 5 |
| 4% by weight 1:1 polyquaternium-53 + | 4 | 5 |
| V02-L2 | | |
| 4% by weight polyquaternium-47 | 1 | 5 |
| 4% by weight 1:1 polyquaternium-47 + | 4 | 4 |
| polycarbodiimide V02-L2 | | |
| 4% by weight polycarbodiimide V02L2 | 5 | 1 |

*amounts of the ingredients in the treatment solutions are expressed in percent total weight of active material in water and ratios are weight ratios

The results in the table above show that the adhesion values for the polyquaternium compounds alone were very poor. The results also show that the conditioning and ease of brushing properties using carbodiimide alone on hair was very poor even though the adhesion value for polycarbodiimide alone was very high. In contrast, the adhesion value of each polyquaternium compound in the table above significantly increased when the polyquaternium compound was combined with a polycarbodiimide. At the same time, this inventive combination resulted in very good conditioning and adhesion properties on hair as evidenced by the values in the table.

5) 2 Step Styling (Blow-Dry)

In a single step (one step) system, a polycarbodiimide and a latex polymer are mixed together and the resulting mixture is applied onto hair (0.2 g/g of hair).

In a two-step system, hair swatches are treated first with a polycarbodiimide solution (0.1 g/g of hair) and then with a latex solution (0.1 g/g of hair).

Hair swatches (IHIP Frizzy Hair) were washed with a cleansing composition.

The test solutions were applied onto the swatches (0.2 g product/g of hair) according to either the single step or two step system. The swatches were rough dried for 30 seconds, then blow-dried on high heat for 1 minute and then placed in a humidity chamber.

TABLE 29

| | | Lasting |
|---|---|---|
| | | frizz in |
| | | Humidity |
| | Initial | Control |
| | frizz in | (5 hr 80% |
| | Humidity | humidity |
| Treatments* | Control | 25° C.) |
| 1 STEP using a combination of 2% by weight of | 4 | 3 |
| polyquaternium 22 and 2% by weight of | | |
| polycarbodiimide V02-L2 (0.2 g of the | | |
| mixture/g of hair) | | |
| 2 Step (0.1 g of 2% by wt polycarbodiimide | 5 | 5 |
| V02-L2/g of hair followed by 0.1 g of 2% by | | |
| weight of polyquaternium 22/g of hair) | | |

*amounts of the ingredients in the treatment solutions are expressed in percent total weight of active material in water and ratios are weight ratios

From the results above, the use of either the one step and the two step systems on the hair resulted in very good frizz control ratings with the ratings being the highest for the two step system with respect to initial and longer lasting frizz control in high humidity environment/temperature.

It is to be understood that the foregoing describes representative embodiments of the disclosure and that modifications may be made therein without departing from the spirit or scope of the disclosure as set forth in the claims.

1. An article of manufacture comprising a kit containing, the kit comprising:
a composition comprising:
(i) at least one polycarbodiimide compound, wherein the polycarbodiimide compound has the following formula: 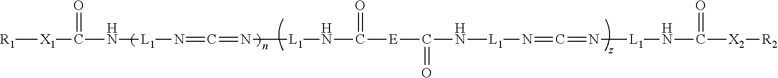
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another;
E is a radical selected from:
O—R₃-0; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms; and
(ii) at least one compound having at least one carboxyl group selected from a cationic polymer compound containing at least one carboxyl group, a carboxylic acid compound chosen from fatty acids, their salts, and mixtures thereof, a polycarboxylic acid, and combinations thereof,
wherein the composition comprises from about 0.1 to about 40%, by weight, based on the total weight of the composition of a combined amount of the polycarbodiimide compound and the cationic polymer compound,
wherein the composition includes amounts of each of the polycarbodiimide compound and the compound having at least one carboxyl group sufficient to impart one or more of increased hydrophobicity to the keratinous substrate after application thereto and increased hold to the shape or configuration of the keratinous substrate
wherein the kit is provided in separate packages selected from
the polycarbodiimide compound and compound having at least one carboxyl group in the composition are combined and the premix comprises at least a solvent; and
the polycarbodiimide compound and compound having at least one carboxyl group in the composition are provided in separate packages, comprising a packaged premix that comprises the polycarbodiimide compound and at least a solvent; and also comprising a packaged premix that comprises the compound having at least one carboxyl group with at least a solvent, whereby at the time of use, the premixed phases are combined and agitated to form the composition.

2. An article of manufacture according to claim 1, the kit comprising:
at least one of:
a separately packaged premix comprising a processing agent selected from a coloring agent, a pigmenting agent, a permanent process agent, a relaxing process agent, a straightening process agent, and a highlighting process agent; and
a separately packaged premix comprising
at least one of the polycarbodiimide compound and at least one of the compound having at least one carboxyl group of a composition according to claim 1, and
at least one processing agent selected from a coloring agent, a pigmenting agent, a permanent process agent, a relaxing process agent, a straightening process agent, and a highlighting process agent.

3. An article of manufacture according to claim 1, wherein any one or more of the premixes comprises at least one additive selected from a solvent chosen from water, organic solvents, and mixtures thereof, and comprising at least one additive chosen from amino silicones, polyamines, diamines, monoamines and amino functionalized silane compounds, anionic surfactants, nonionic surfactants, cationic surfactants, amphoteric surfactants, zwitterionic surfactants), anionic polymers, nonionic polymers, amphoteric polymers, polymeric rheology modifiers, thickening agents viscosity modifying agents, associative thickeners, non-associative polymeric thickeners, non-polymeric thickeners, nacreous agents, opacifiers, dyes, pigments, fragrances, mineral, plant oils, synthetic oils, waxes, ceramides, vitamins, UV-screening agents, free-radical scavengers, antidandruff agents, hair-loss counteractants, hair restorers, preserving agents, pH stabilizers, and mixtures thereof.

4. An article of manufacture according to claim 1, wherein each of the polycarbodiimide compound and the compound having at least one carboxyl group is present in a concentration, by weight, of from about 0.01% to about 10%.

5. An article of manufacture according to claim 1, comprising an additive selected from one or a combination of carboxysilicone polymers, amino compounds, amino silicone compounds, and latex polymers.

6. An article of manufacture according to claim 1, wherein the compound having at least one carboxyl group includes a cationic polymer compound containing at least one carboxyl group selected from the group consisting of polyquatemium 22, polyquatemium-39, polyquatemium-47, polyquatemium-53); polyquaternium-30, polyquatemium-35, polyquaternium-45, polyquatemium-50, polyquaternium-54; polyquatemium-57; polyquaternium-63; polyquatemium-74; polyquaternium-76; polyquatemium-86; polyquatemium-89; polyquatemium-95; polyquatemium-98, polyquatemium-104; polyquaternium-111; polyquatemium-112, and mixtures thereof.

7. An article of manufacture according to claim 6, wherein the cationic polymer compound containing at least one carboxyl group is selected from the group consisting of polyquaternium 22, polyquatemium-39, polyquaternium-47, and polyquaternium-53.

8. An article of manufacture according to claim 1, wherein the hydrophobicity imparted to hydrophilic keratinous substrates confers one or more of frizz control at conditions of high humidity, ease of distribution, ease of brushing, and adhesion.

9. An article of manufacture according to claim 8, wherein the hydrophobicity imparted to hydrophilic keratinous substrates confers conditioning, ease of brushing and good adhesion

10. An article of manufacture according to claim 1, wherein the combination of polycarbodiimide compound and compound having at least one carboxyl group is present in an amount, by weight, in the range from about 1% to about 5%, and at a ratio of polycarbodiimide and compound having at least one carboxyl group, from about 1:10 to about 10:1.

11. An article of manufacture according to claim 10, wherein the ratio of polycarbodiimide compound and the compound having at least one carboxyl group is about 1:1.

12. An article of manufacture according to claim 10 wherein the compound having at least one carboxyl group is a cationic polymer compound containing at least one carboxyl group that comprises one or more or of polyquaternium-22, polyquaternium-39, polyquaternium-47, and polyquaternium-53.

13. A method of protecting a keratinous substrate chosen from hair, eyelashes and eyebrows from extrinsic damage caused by heating, UV radiation, chemical treatment or mechanical stress, or of repairing a keratinous substrate chosen from hair, eyelashes and eyebrows following extrinsic damage caused by heating, UV radiation, chemical treatment, or mechanical stress comprising:
applying to the keratinous substrate a composition in an amount effective to protect or repair the keratinous substrate; wherein the polycarbodiimide compound is present at a concentration of from about 0.25 to about 20.0% by weight, relative to the total weight of the composition, the composition comprising
(i) at least one polycarbodiimide compound, wherein the polycarbodiimide compound has the following formula: 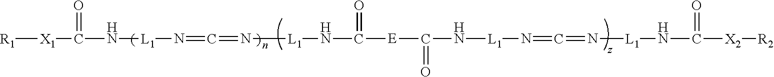
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another;
E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms; and
(ii) at least one compound having at least one carboxyl group selected from a cationic polymer compound containing at least one carboxyl group, a carboxylic acid compound chosen from fatty acids, their salts, and mixtures thereof, a polycarboxylic acid, and combinations thereof,
wherein the composition comprises from about 0.1 to about 40%, by weight, based on the total weight of the composition of a combined amount of the polycarbodiimide compound and the at least one compound having at least one carboxyl group,
wherein the composition includes amounts of each of the polycarbodiimide compound and the at least one compound having at least one carboxyl group sufficient to impart one or more of increased hydrophobicity to the keratinous substrate after application thereto and increased hold to the shape or configuration of the keratinous substrate.

14. The method of claim 13, further comprising: applying heat to the keratinous substrate wherein the heat is applied prior to or while or after applying the composition to the keratinous substrate.

15. The method of claim 13, wherein the method imparts hydrophobicity to hydrophilic keratinous substrates that confers high humidity curl retention after 5 hours of exposure in the range from about 30% to about 90%.

16. The method of claim 13, wherein the method imparts hydrophobicity to hydrophilic keratinous substrates that confers a bending force property to the keratinous substrate.

17. The method of claim 13, wherein the method imparts hydrophobicity to hydrophilic keratinous substrates that confers lastingness characterized by a contact angle of from about 500 to about 120° after washing and blow drying.

18. The method of claim 13, wherein the method imparts increased hold to the shape or configuration of the keratinous substrate which confers improved bending force property to the keratinous substrate.

19. A method for durable non-permanent shaping of at least one keratinous substrate or for durable retention of a non-permanent shape of at least one keratinous substrate comprising: applying to the at least one keratinous substrate a composition; optionally heating the at least one keratinous substrate; wherein when heating is employed, the composition is applied prior to the heating or during the heating or after the heating, the composition comprising
(i) at least one polycarbodiimide compound, wherein the polycarbodiimide compound has the following formula: 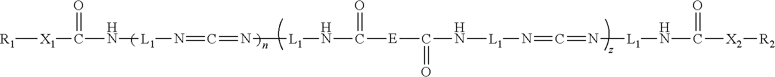
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms; and
(ii) at least one compound having at least one carboxyl group selected from a cationic polymer compound containing at least one carboxyl group, a carboxylic acid compound chosen from fatty acids, their salts, and mixtures thereof, a polycarboxylic acid, and combinations thereof,
wherein the composition comprises from about 0.1 to about 40%, by weight, based on the total weight of the composition of a combined amount of the polycarbodiimide compound and the compound having at least one carboxyl group,
wherein the composition includes amounts of each of the polycarbodiimide compound and the compound having at least one carboxyl group sufficient to impart one or more of increased hydrophobicity to the keratinous substrate after application thereto and increased hold to the shape or configuration of the keratinous substrate.

20. A method of protecting a keratinous substrate chosen from hair, eyelashes and eyebrows from extrinsic damage caused by heating, UV radiation, chemical treatment or mechanical stress, or of repairing a keratinous substrate chosen from hair, eyelashes and eyebrows following extrinsic damage caused by heating, UV radiation, chemical treatment, or mechanical stress comprising at least one polycarbodiimide compound and at least one compound having at least one carboxyl group, wherein the composition is provided in at least one premix comprising at least a solvent, and the at least one premix is applied to the keratinous substrate according to a process selected from a one step process and a two-step process,
(a) the composition comprising
(i) at least one polycarbodiimide compound, wherein the polycarbodiimide compound has the following formula: 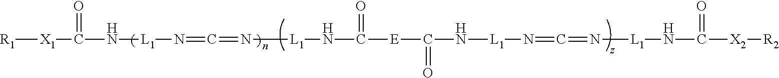
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms; and
(ii) at least one compound having at least one carboxyl group selected from a cationic polymer compound containing at least one carboxyl group, a carboxylic acid compound chosen from fatty acids, their salts, and mixtures thereof, a polycarboxylic acid, and combinations thereof,
wherein the composition comprises from about 0.1 to about 40%, by weight, based on the total weight of the composition of a combined amount of the polycarbodiimide compound and the compound having at least one carboxyl group,
wherein the composition includes amounts of each of the polycarbodiimide compound and the compound having at least one carboxyl group sufficient to impart one or more of increased hydrophobicity to the keratinous substrate after application thereto and increased hold to the shape or configuration of the keratinous substrate.
(b) wherein the process is at least a one step process selected from
a one step process, wherein the composition comprising the polycarbodiimide compound and the compound having at least one carboxyl group is provided as a premix, and is prepared by combining the polycarbodiimide compound and the compound having at least one carboxyl group and at least a solvent, whereby the premixed composition is applied onto the keratinous substrate;
a one step process, wherein the composition is provided in separate premixes, each separately comprising the polycarbodiimide compound and the compound having at least one carboxyl group, the premixes prepared by combining the polycarbodiimide compound with at least one solvent to form on first phase, and separately combining the compound having at least one carboxyl group with at least one solvent to form a second phase, whereby at the time of use, the premixed phases are combined and to form a composition that is applied onto the keratinous substrate; and
a two-step process, wherein the composition is provided in separate premixes, the premixes prepared by combining the polycarbodiimide compound with water to form on a first phase, and separately combining the compound having at least one carboxyl group with a solvent chosen from one or more of water and an organic solvent to form a second phase, whereby at the time of use, the each of the premixed phases is applied separately to the keratinous substrate in any order.

21. The method of claim 20, wherein the process comprises, in any order, one or more additional process steps selected from
a step of applying heat to the keratinous substrate wherein the heat is applied prior to or while or after applying any one or more premixed composition to the keratinous substrate; and
a step of processing the keratinous substrate by any one or more of processes selected from coloring, pigmenting, perming, relaxing, straightening, and highlighting.

22. The method of claim 20, wherein in any combination of two or more process steps, any one or more of the premix compositions is provided for application together with any one of a coloring agent, a pigmenting agent, a permanent process agent, a relaxing process agent, a straightening process agent, and a highlighting process agent.

23. The method of claim 20, wherein at least one premix that comprises one or both of the polycarbodiimide compound and the compound having at least one carboxyl group also comprises one of the coloring agent, a pigmenting agent, a permanent process agent, a relaxing process agent, a straightening process agent, and a highlighting process agent.
